(19) United States
(12) Patent Application Publication
(10) Pub. No.: US 2016/0354034 A1
(43) Pub. Date: Dec. 8, 2016

(54) SYSTEMS AND METHODS FOR REDUCING SCAR FORMATION ABOUT A NEURAL IMPLANT

(71) Applicant: Massachusetts Institute of Technology, Cambridge, MA (US)

(72) Inventors: Kevin C. Spencer, Cambridge, MA (US); Jay Christopher Sy, Chelsea, MA (US); Michael J. Cima, Winchester, MA (US)

(21) Appl. No.: 15/172,464

(22) Filed: Jun. 3, 2016

Related U.S. Application Data
(60) Provisional application No. 62/171,055, filed on Jun. 4, 2015.

(51) Int. Cl. A61B 5/00; A61M 37/00; A61N 1/05; A61B 5/04; A61B 5/0476

(52) U.S. Cl. CPC A61B 5/686; A61B 5/04001; A61B 5/0476; A61N 1/0551; A61N 1/0536; A61M 37/00

(57) ABSTRACT

Systems, devices, and methods are provided for reducing scar formation about a neural implant due to brain tissue and neural implant movement. In an embodiment, a neural implant is provided which has a surface coating that matches one or more mechanical properties, such as elastic modulus, of the brain tissue, thereby reduce scar formation about the neural implant due to normal brain micromotion.

### CROSS-REFERENCE TO RELATED APPLICATIONS

This disclosure claims priority to and the benefit of U.S. provisional patent application No. 62/171,055, filed Jun. 4, 2015, which is incorporated herein by reference in its entirety.

### STATEMENT REGARDING FEDERALLY SPONSORED RESEARCH OR DEVELOPMENT

This invention was made with government support under Grant No. R01 EB016101 awarded by the National Institutes of Health and National Institute of Biomedical Imaging and Bioengineering. The government has certain rights in the invention.

### FIELD

This disclosure generally relates to medical devices and more particularly relates to systems and methods for reducing scar formation about a neural implant due to brain tissue and neural implant movement.

### BACKGROUND

Some neurological diseases and disorders involve errors in the transmission of chemical and electrical signals. These errors can lead to miscommunication between different regions of the brain and cause circuit disorders. Treatments include deep brain stimulation, which has proven to be effective for several disorders. This surgical treatment allows scientists to electrically modulate and record brain activity in target regions by implanting an electrode into specific areas of the brain.

Neural implants have been developed to accomplish a wide range of functions, including, but not limited to, local stimulation, recording of neural activity, and/or the delivery of pharmaceutical compounds. A large problem with device implantation into the brain is the brain tissue's response to these implants. Research has suggested that the surrounding tissue will initiate an inflammatory and wound healing response that is exacerbated over time as a result of the brain's micro-motions from everyday movement. A fibrous capsule develops over time that interferes with electrode and neuron cell body contact, which can negatively impact device function.

A large contributor to neural implant failure is the formation of glial scar tissue around the site of implantation. Glial scar formation increase tissue impedance and restrict the transport of drugs. The glial scar manifests as a dense sheath surrounding the implant and is a result of astrocytes and microglia reacting with the foreign material. As a result, the effectiveness and/or utility of neural implants are limited in chronic settings due to glial scar formation. Accordingly, there is a need for a neural implant with increased biocompatibility that is configured to diminish the incidence and extent of glial scar formation, thus prolonging the functionality of the neural implant.

U.S. Pat. No. 8,798,737 discloses one approach to improve the interface between electrodes and neural tissues by utilizing a biodegradable coating to release an anti-inflammatory drug. Lu et al., Biomaterials 30(25): 4143-51 (2009) discloses the use of a hydrogel poly(vinyl alcohol)/poly(acrylic acid) interpenetrating polymer network as a coating for poly(dimethylsiloxane) based neural electrodes with the aid of plasma pretreatment to reduce protein absorption. Improved neural implant devices and methods are still needed to diminish the incidence and extent of glial scar formation, preferably without the administration of drug.

### SUMMARY

Some or all of the foregoing needs and/or problems may be addressed with one or more of the embodiments of the present disclosure. In certain embodiments, a medical device for insertion into brain tissue is disclosed. The medical device includes a neural implant. A coating is disposed on a surface of the neural implant. The coating possesses mechanical properties that substantially match one or more mechanical properties of the brain tissue to reduce scar formation about the neural implant due to brain tissue and neural implant movement.

In one aspect, a medical device is provide for use in brain tissue, wherein the device includes a neural implant configured for insertion into brain tissue; and a coating disposed on a surface of the neural implant, wherein the coating is configured to exhibit in vivo (i) an elastic modulus that substantially matches the elastic modulus brain tissue and (ii) a thickness to accommodate micromotions of the brain tissue relative to the neural implant.

In another aspect, a system is provided for delivering chemical and/or electrical stimulation across one or more neural circuits and/or to sense and record neural activity, wherein the system includes an elongated, rigid neural implant insertable into the brain tissue; and a hydrogel coating disposed on a surface of the neural implant, wherein the hydrogel coating which is configured to exhibit in vivo (i) an elastic modulus that substantially matches the elastic modulus brain tissue and (ii) a thickness to accommodate micromotions of the brain tissue relative to the neural implant. In exemplary embodiments, the hydrogel coating has an elastic modulus of about 5 kPa to about 300 kPa, a thickness of from about 15 micron to about 500 microns, or a combination of these characteristics. In one embodiment, the surface of the neural implant is cylindrical and the hydrogel coating covers the full circumference of the cylindrical surface about substantially the full length of the neural implant insertable into the brain tissue.

Other features and aspects of the disclosure will be apparent or will become apparent to one with skill in the art upon examination of the following figures and the detailed description. All other features and aspects, as well as other system, method, and assembly embodiments, are intended to be included within the description and are intended to be within the scope of the accompanying claims.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIGS. 1A and 1B depict possible mechanics of cellular response to a neural implant during acute and chronic phases, respectively.

FIGS. 2A-2D depict the formation of glial scar around a neural implant over time.

FIG. 3A depicts hydrogel coating thickness as a function of the number of spray coats.

FIG. 3B depicts the elastic modulus as a function of polymer concentration (w/w) in phosphate buffered saline before crosslinking.

FIGS. 4A-4C depict hydrogel coated devices resulting from spray coating, cast molding, and dip coating, respectively.

FIG. 5A depicts a coated device before and after dehydration.

FIG. 5B depicts the swelling kinetics in water and in an agarose tissue phantom.

FIG. 6 depicts a process of surface functionalization of glass substrates.

FIG. 7 depicts a schematic representation of a PEGDM network.

FIG. 8 depicts a strain field measurement experimental setup.

FIG. 9 depicts an optical bright field microscope image of an uncoated device.

FIG. 10 depicts a plot of modulus vs. molecular weight of poly(ethylene glycol) (PEG).

FIG. 11 depicts a plot of modulus vs. concentration of PEG.

FIG. 12 depicts a PEG-2000 coated device.

FIGS. 13A-13C depict vector plots of particle displacement field.

FIGS. 14A-14C depicts particle displacement field vector plot overlays.

FIG. 15A depicts a particle displacement field cross section of a control device.

FIG. 15B depicts a particle displacement field cross section of PEG-8000 device.

FIG. 16 depicts a particle displacement field cross section of PEG-2000 device.

FIG. 17 depicts displacement at the surface of various coated and uncoated devices.

FIG. 18 illustrates a photo-polymerization process for generating free radicals to initiate a polymerization process.

FIG. 19 depicts the preparation of hydrogel coating on a capillary tube.

FIG. 20 depicts a plot of molecular weight between crosslinks vs. molecular weight of PEG.

FIG. 21 depicts a plot of elastic modulus vs. molecular weight of PEG.

FIG. 22 depicts cross sectional views of a hydrogel coated capillary tube as initially applied, as dried, and as rehydrated.

FIGS. 23A-23C depict various side views of a hydrogel coated capillary tube in an initial swollen state, a dried state, and in a rehydrated and swollen state, respectively.

FIGS. 24A and 24B depict the insertion of a swollen hydrogel into the brain phantom, which caused the hydrogel coating to shear off the capillary tube.

FIG. 25 depicts a plot of re-swelling ratio vs. time for a 10% 700 molecular weight hydrogel coated capillary tube in water or agarose.

FIG. 26 depicts a plot of re-swelling ratio vs. time for a 20% 700 molecular weight hydrogel coated capillary tube in water or agarose.

FIG. 27 depicts a plot of re-swelling ratio vs. time for a 20% 700 molecular weight hydrogel coated capillary tube in water or agarose over the first 125 seconds.

FIG. 28A depicts a hydrogel coated capillary tube with the direction of implementations into agarose.

FIG. 28B depicts a hydrogel coated capillary tube after 300 seconds of swelling in agarose.

FIG. 28C depicts a sample vector plot for the time interval of 2-4 seconds with white lines indicating the capillary and gel boundaries.

FIGS. 28D-28H depict the displacement values of agarose over various time intervals.

FIG. 29 depicts buckling of hydrogel during swelling following drying of the hydrogel coating on capillary tube over an extended period.

FIG. 30A depicts representative fluorescent images for coated and uncoated devices in an animal model.

FIG. 30B depicts quantification of the GFAP expression as a function of distance from the implant at one week post implantation.

FIG. 31A depicts representative fluorescent images for coated and uncoated devices in an animal model.

FIG. 31B depicts quantification of the GFAP expression as a function of distance from the implant at four week post implantation.

### DETAILED DESCRIPTION

Systems, devices, and methods have been developed for reducing scar formation (e.g., glial scar formation) about a neural implant due to brain tissue and neural implant movement.

In one aspect, a neural implant is provided which incorporates materials with lower mechanical strengths or coatings that promote adhesion with neural tissue to reduce the extent of glial scar formation. For example, conformal polymer hydrogel coatings are disclosed herein that substantially match the mechanical properties (e.g., the elastic modulus) of brain tissue. Methods to covalently bind these coatings to the surface of a neural implant are also disclosed. This material system and coating method result in a surface of the neural implant which more closely matches the mechanical properties of brain tissue.

The elastic modulus and dimensions of the coating may be controlled by adjusting the polymer composition of the hydrogel coating. In some instances, the coatings advantageously are dry prior to implantation (minimizing implantation damage and ensuring coating integrity) and swell to form a hydrogel once implanted in the body. The hydrogel coating beneficially reduces the overall effective modulus of the neural implant. This reduces the scar formation around the neural implant by minimizing damage from micromotion, and helps contribute to proper long term implant function.

The Neural Implant

The systems and methods disclosed herein can be adapted for use with essentially any medical device configure for placement in contact with brain tissues or other neurological tissues over an extended period. In one embodiment, the medical device is a neural implant as known in the art, which may be configured to deliver chemical and/or electrical stimulation across one or more neural circuits and/or may be configured to sense and record neural activity. Essentially any type of neural implant may be used herein. The neural implant may include a narrow elongated body portion, which may be substantially rigid and may include one or more channels for carrying electrodes, sensors, fluids for injection or withdrawal, or combinations thereof. In various embodiments, the neural implant is an injection-microelectrode (“injectrode”), a micro-injectrode, or the like. The neural implant may comprise a cannula. In some instances, the neural implant includes a chronic intracranial implant configured to facilitate the administration of chemical and/or electrical therapy to specific anatomical (e.g., brain) nuclei. In some instances, the neural implant may be a passive device, an active device, or a combination thereof.

The neural implant generally is configured to be partially or fully implantable in a patient. The patient may be human or other mammal. In order to effectively treat neural circuit disorders, the devices such as the neural implants often require chronic operation. A suitable neural implant as described herein should operate effectively at the time of implantation and continue to do so in the presence of bodily biological responses to the device.

Understanding Glial Scar Formation

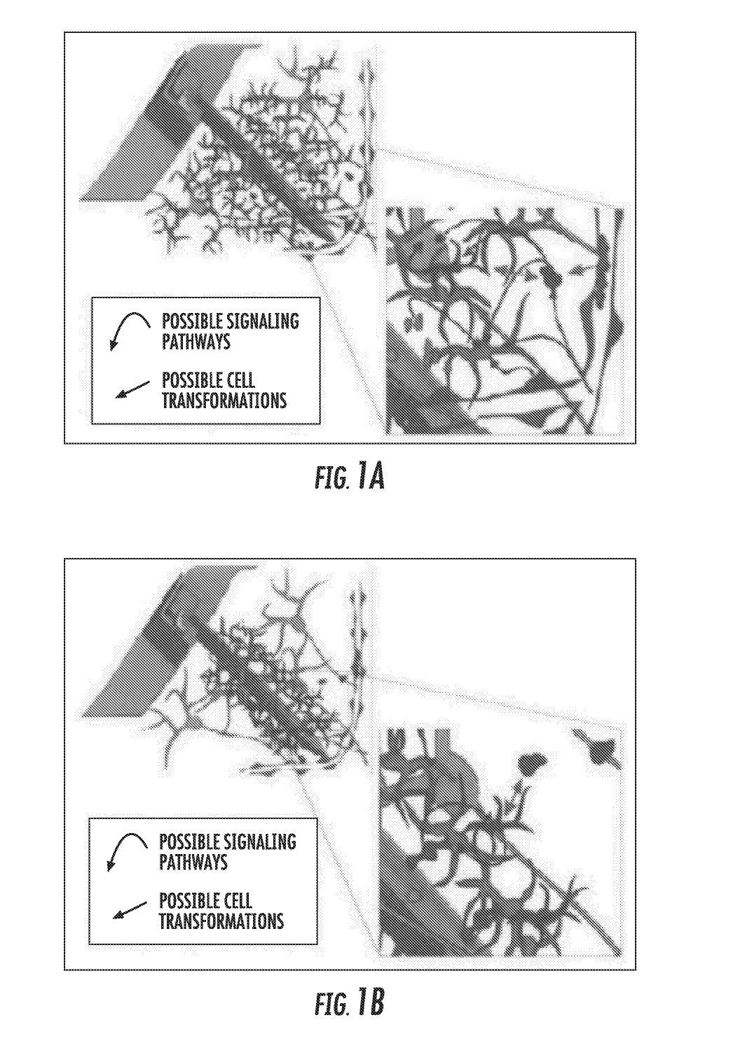

The brain's primary immune response to neural implants, astrogliosis, results in the formation of glial scar tissue around the neural implant. The main cellular contributors in the astrogliosis response to implants or other injury to the central nervous system are astrocytes and microglia. Astrocytes, characterized by immunostaining for the intermediate filament protein glial fibrillary acidic protein (GFAP), are the main component of glial scar tissue. Microglia are macrophages in the brain that engulf particles and secrete proteins that affect a variety of processes that support inflammation. Astrogliosis is generally divided into two main phases, the acute phase and the chronic phase. FIG. 1A depicts possible mechanics of cellular response to an implant during the acute phase, and FIG. 1B depicts possible mechanics of cellular response to an implant during the chronic phase.

The short-term response to foreign object implantation in the central nervous system is what is referred to as the acute response. In some instances, the acute response occurs over about the first two weeks of implantation and is characterized by a large amount of microglia activity. Implantation of the device causes injury to the brain through cell damage, blood vessel severing, and disruption of the extracellular matrix. This injury causes the release of several protein factors into the area to promote inflammation and clotting. The proteins promoting inflammation cause inflammatory cells and microglia to join and form an envelope of cells around the injury. The microglia then breakdown red blood cells in the area and any cellular remains from the injury, then release cytokines and reactive oxygen intermediates in order to promote inflammation. This inflammatory response can negatively affect neural implant effectiveness in the area of the injury.

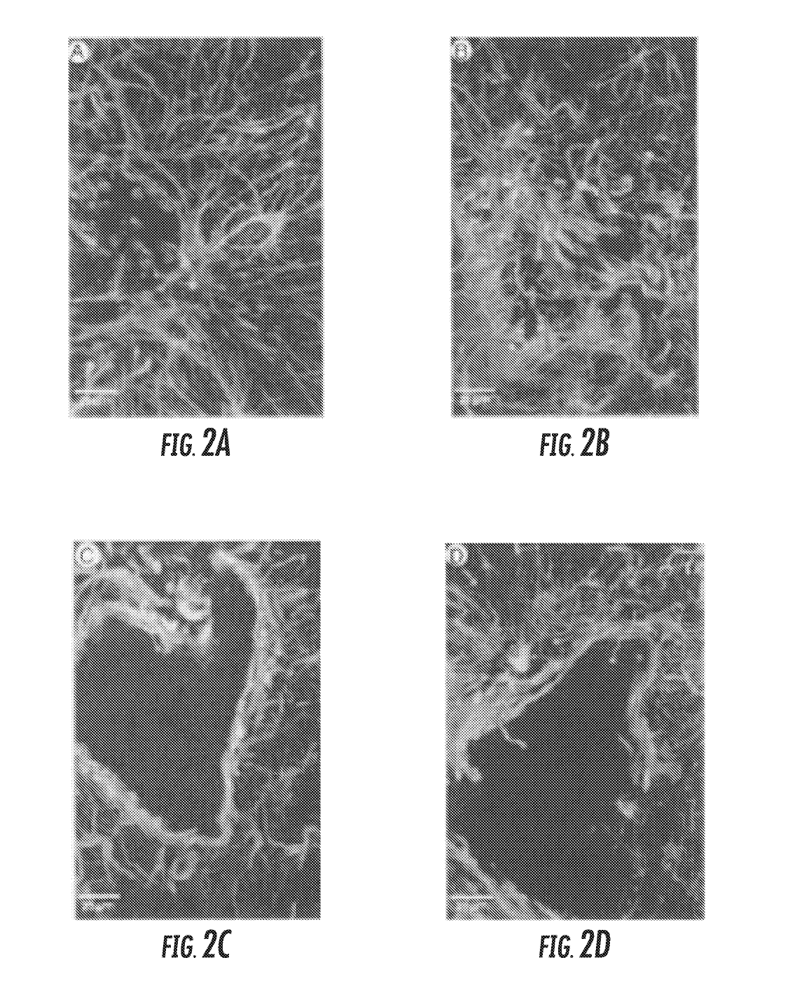

Implantation times longer than two weeks usually mark the end of the acute phase and lead to the chronic phase of the astrogliosis. The chronic phase is characterized by the formation and densification of glial scar tissue. Astrocytes isolate the injury site from the rest of the neural tissue even further than was done in the acute phase and increase production of certain extracellular molecules, including chondroitin sulfate proteoglycans that inhibit axon regeneration. The glial tissue, typically a few hundred microns thick, typically reaches its full size 4-6 weeks after the initial injury and remains generally stable for the duration of the implantation. FIGS. 2A-2D depict glial scar formation around a neural implant over time. For example, FIG. 2A depicts glial scar formation around a neural implant after 2 weeks, and FIG. 2B depicts glial scar formation around a neural implant after 4 weeks. At 2 and 4 weeks, the glial scar falls back into the void left by the electrode. FIG. 2C depicts glial scar formation around a neural implant after 6 weeks, and FIG. 2D depicts glial scar formation around a neural implant after 12 weeks. At 6 and 12 weeks, the glial scar is a dense sheath which does not collapse into the void.

This process of the chronic phase is analogous to the fibrous encapsulation observed around implants in other parts of the body. Additionally, microglia typically remain at the surface of the implant injury throughout the chronic phase forming a dense layer of cells on the implant surface that is surrounded by the glial scar.

Glial scar is formed with the intent to protect the brain and the rest of the central nervous system from the implanted foreign body within the tissue as well as the reactive proteins and molecules released to the injury site in the process. However, while generally beneficial, glial scar formation is believed to be a key contributor to neural implant failure. For example, nearly half of conventional recording electrode implants fail 6 months after implantation despite initially operating properly. Neurons typically need to be less than 100 μm from the recording electrode to be recorded, and the formation of glial scarring forces neurons near the implant away from the injury site, decreasing the potential signal that can be recorded by these devices and eventually leading to complete failure of the electrode implant. Glial scarring increases the impedance of the tissue, thus reducing the volume of tissue activated by electrical stimulation by up to 50%. This can be partially overcome to get the desired therapeutic effect on a given neural circuit through use of a higher current but a higher current can lead to neuron damage.

Diffusion properties of cells change following astrogliosis in response to a stab wound. For example, the astrogliosis leads to an increase in tortuosity, increase in the volume fraction of extracellular space, and decreased cellular uptake. This hindered diffusion environment likely affects the extra synaptic transmission of neurons, hinders diffusion of glucose and oxygen to cells from circulation, and contributes to the barrier to axon regeneration around the injury. It should be noted that the glial scar resulting from stab wounds should have slightly different properties than those resulting from chronic implants due to constant presence of brain micromotion in the case of chronic implants.

Brain tissue is constantly undergoing micromotions. For example, in rats, brain tissue may move (relative to the position of, and within, the skull) up to about 40 μm in magnitude due to respiration, vascular pulses, and rotational accelerations. Similar brain tissue movement occurs in humans.

As depicted in Table 1, typical neural implant device materials of construction have moduli that are many orders of magnitude higher than that of brain tissue.

TABLE 1

| | Elastic |
|---|---|
| | Modulus |
| Material | (GPa) |
| Borosilicate Glass | 69 |
| Gold | 80 |
| Silicon | 200 |
| Titanium | 107 |
| Polyimide | 2 |
| Brain | .000005 |

Constant relative motion between the neural implant and tissue is thought to play a key role in the chronic tissue response through constant aggravation of local inflammatory cells and damage to local vasculature. Indeed, implants that are tethered to the skull result in a much greater glial scar response than untethered implants which are free to move with the brain's micromotion. Tethered implants are fixed in place relative to the skull and thus result in a greater relative motion between the implant and brain tissue. This results in greater injury and aggravation of tissue surrounding the implant and more extensive scar formation. Finite element analysis simulations have been conducted to investigate the effect that micromotion and mechanical mismatch have on the surrounding tissue. These simulations estimate the amount of strain that the brain tissue experiences as a result of brain micromotion in the presence of neural implants of various mechanical properties. It was found that a probe composed of a hypothetical soft material with Young's modulus of 6 MPa results in a strain two orders of magnitude less than that of a silicon probe with a modulus 200 GPa. Poor tissue-device adhesion also was found to contribute to elevated strains. A tangential tethering force from tissue adhesion to the device reduced strains near the tip of the implant by 94%. This further corroborates the theory that tissue aggravation causes glial scar formation.

Experiments have demonstrated a direct correlation between induced strains at a probe-tissue interface and the resultant scarring, suggesting mechanical mismatch to be a cause of scar formation. As noted above, the elastic modulus of neural implants is often orders of magnitude larger than that of the brain. Borosilicate glass and the brain, for example, have elastic moduli of about 69 GPa and 5 kPa, respectively. This mismatch causes the soft brain tissue to experience the majority of the strain. When the modulus of the implant is reduced, more strain will be shared between the device and the brain tissue. Finite element analysis indicates that reducing the moduli of the implant to the scale of megapascals can reduce the strain on the brain by two orders of magnitudes. This demonstrates a need for mechanical compatibility between the device and the brain tissue to minimize the brain's immune defenses and promote long-term usage of the device.

Glial scar tissue forms in the brain as a response to the implant injury and hampers the effectiveness of the implant treatment. Constant relative micromotion between the mechanically mismatched neural implant and brain tissue is thought to play a role glial scar formation. That is, the discrepancy in mechanical properties of traditionally used implant materials (e.g., elastic modulus on the order of GPa) vs. brain tissue (e.g., elastic modulus of about 5 kPa) is a key contributor to scar formation. The brain tissue undergoes micromotion displacements due to head acceleration as well as respiration/vascular pulsations which lead to local strain around the implant. This aggravates local glial cells and leads to an increased scar formation, impairing the long term function of the neural implant. Biochemical coatings incorporated into the neural implant may be used to diminish the incidence and extent of glial scar formation, thus prolonging the functionality of the neural implant.

Mechanically Matched Coating

It was therefore determined that the mechanical properties of a hydrogel coating can have a substantial influence on the biocompatibility of a neural implant device. As described herein, in order to accommodate the micromotion and otherwise reduce the gliosis response, neural implant devices are provided with a coating that is substantially matched to the mechanical properties of the tissues at the implantation site. In a preferred embodiment, the coating is a polymeric conformal coating. In one embodiment, the coating is a hydrogel. The conformal coating may be over part of or the entire device structure that is in contact with brain tissues.

In certain embodiments, the coating is provided with an elastic modulus that substantially matches that of brain tissue at the implantation site. For example, the mechanical properties of brain tissue vary, based for example on brain region, from about 5 kPa to about 100 kPa. In one embodiment, the neural implant device has a hydrogel coating with moduli between 5 kPa and 300 kPa. Any suitable modulus of the coating may be used.

Importantly, in addition to the modulus, the thickness of the coating is selected to accommodate the elastic deformation associated forces applied to the coating from brain micromotion. Typical brain micromotion is on the order of 30 microns. Accordingly, in one embodiment, the thickness of the swollen hydrogel coating is at least 15 microns (where for example two opposing sides are coated providing a total, combined thickness of 30 microns). For example, the swollen hydrogel coating thickness may range from 20 microns to 500 microns, e.g., from 30 microns to 500 microns, from 40 microns to 400 microns, from 50 microns to 500 microns, from 50 microns to 400 microns, from 50 microns to 300 microns, or from 50 microns to 250 microns. The swollen hydrogel coating thickness may be any suitable thickness. The thickness may be uniform or vary. It is noted, however, that a swollen hydrogel coating having a total thickness, along one axis of motion, less than 30 microns may undesirably fail accommodate the brain micromotion. That is, if the coating is too thin, the hydrogel coating may be unable to elastically deform by 30 microns in one direction and thereby may be unable to absorb the full range of motion of the brain.

Implanting a neural device having a hydrogel coating results in a reduced reactive gliosis response compared to uncoated devices following micromotion. Even if the presence of a hydrogel coating may not completely prevent scar formation due to the central nervous system's foreign body response, a mechanically matched coating may reduce injury to the brain tissue from micromotion induced strain. This decrease in strain imposed on the brain reduces the astrogliosis response and the overall size of the glial scar is reduced, increasing the effectiveness of the neural implant.

The mechanical properties of a hydrogel can be tuned to match that of brain tissue. The modulus of the coating may be controlled by adjusting the polymer chemistry and concentration before crosslinking. The swollen thickness of the coatings may be controlled by making adjustments to the coating protocol used. The flexibility of varying coating parameters enables one to optimize the formulation based on a desired application. The coating mechanical strength may be chosen to match the surrounding tissue.

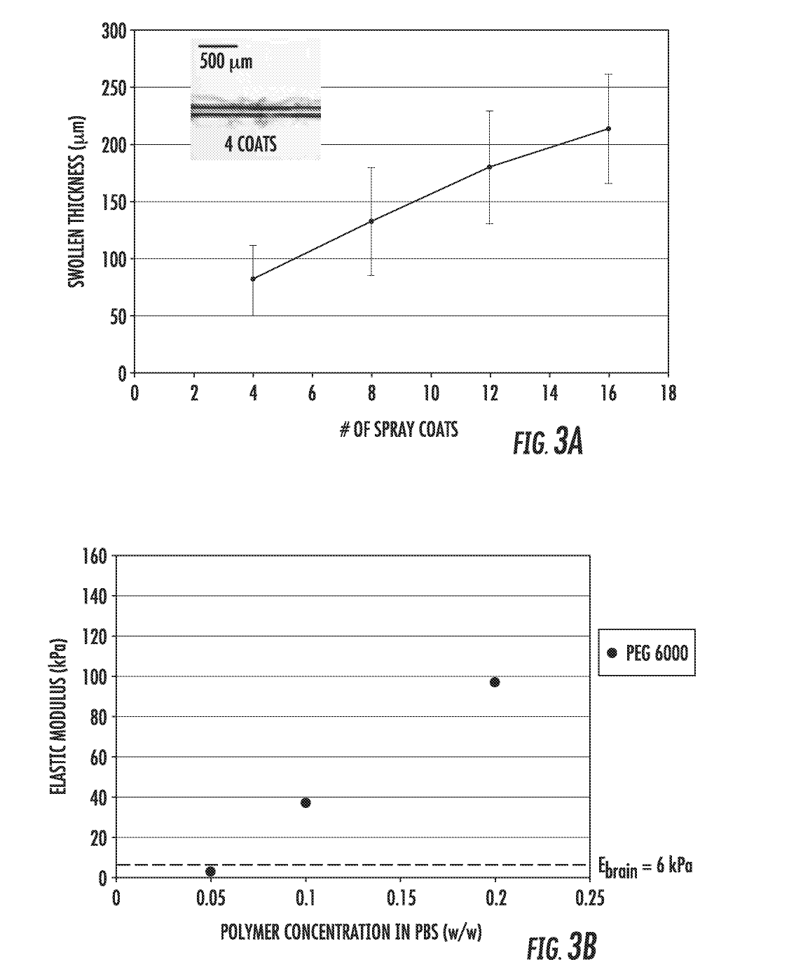

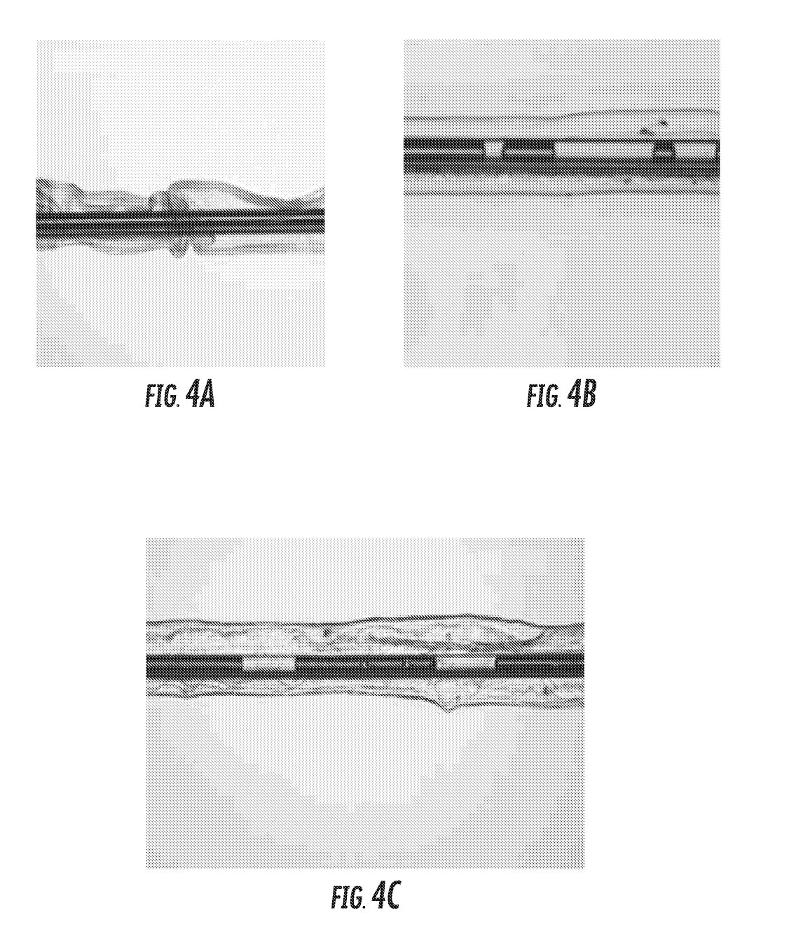

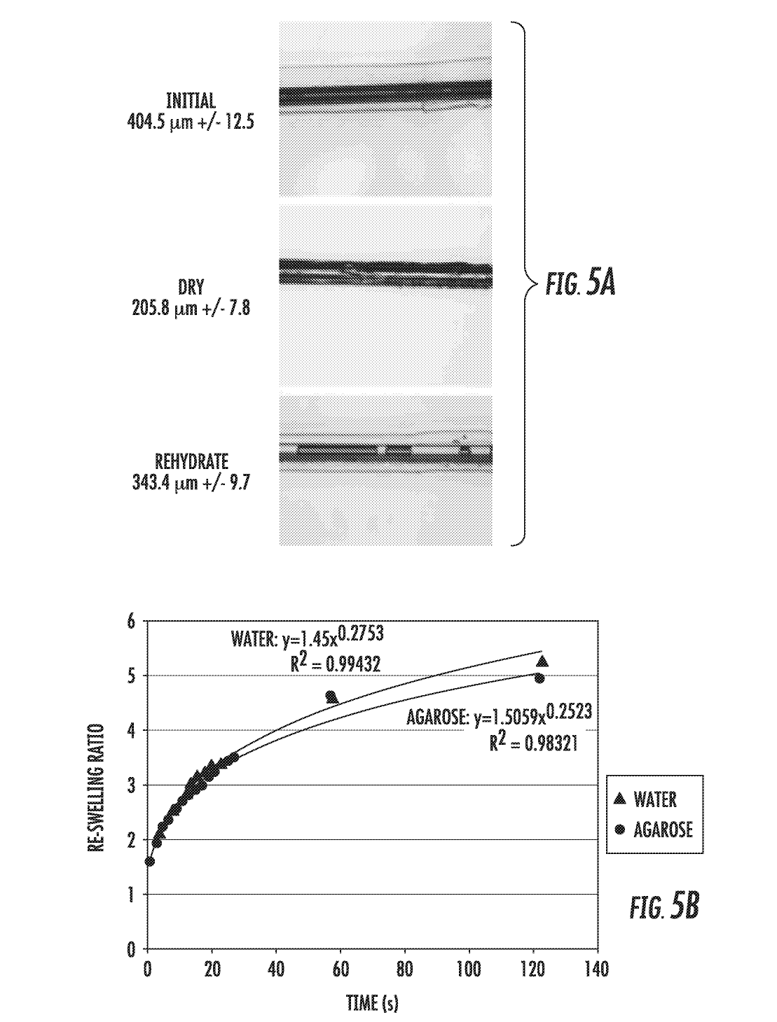

The conformal coatings may be applied via spray coating, cast molding, dip coating, or a combination thereof. FIG. 3A depicts the coating thickness as a function of the number of sprays, and FIG. 3B depicts the modulus as a function of polymer concentration before crosslinking. As depicted in FIGS. 4A-4C, neural devices of any geometry may be coated. The neural devices may be spray coated (as depicted in FIG. 4A), cast molded (as depicted in FIG. 4B), or dip coated (as depicted in FIG. 4C) with a hydrogel or the like. Moreover, as depicted in FIG. 5A, and discussed in greater detail below, to prevent damage from implantation, the coatings may be dried prior to implantation and then re-swollen to their original dimensions following implantation in the brain tissue. For example, FIG. 5A depicts a coated device before and after dehydration. The gel was then inserted into an agarose tissue phantom and the coating rehydrated. The swelling kinetics in water and tissue phantom are shown in FIG. 5B.

In one embodiment, the molecular weight of the gel precursor polymer chains and the concentration of those monomers in solution are controlled/selected to provide the desired coating specification.

The microstructural origin of gel elastic response is the entropic spring effect. Stretching polymer chains, or otherwise mechanically forcing them out of a high-entropy coil conformation, reduces the number of available microstructural conformations available to that chain, decreasing the entropy of the system. Since the potential entropic contribution to the free energy of the chains is so high, the stretched state may be thermodynamically unfavorable. When the external stretching force is removed from the system, the polymer chain will snap back to its equilibrium, unstretched state.

Hydrogels are formed by linking together many smaller chains of a water-soluble polymer. One method of crosslinking chains is through radiation crosslinking using ultraviolet light. Irradiation of polymer containing solution causes the creation of free radicals which recombine to form chemical crosslinks between chains. The resulting gel can then swell in the presence of a solvent, which in the case of hydrogels, is water. The Young's modulus of these gels is highly dependent on the total crosslink density which includes both chemical and physical crosslinks. Increasing the crosslink density restricts chain movement within the network. As a consequence, this prevents deformation of the overall structure in response to applied stress.

Crosslinking density is primarily tunable through selection of gel precursor. Free radical formation, the first step in chemical crosslinking, occurs at the ends of polymer chains. Increasing the density of polymer chains ends before gelation would therefore increase the resulting chemical crosslink density. The simplest way of increasing chain ends is using small molecular weight chains as a precursor. Smaller molecular weight means fewer monomers per chains and a shorter chain, meaning an increase in chain ends per monomer. Gels may be formed from high molecular weight chains or low molecular weight chains, and the chemical crosslink density is much higher in the lower molecular weight case.

Physical crosslinks occur when the polymer chains get entangled and restrict movement. This is purely a topological constraint imposed on the system when two polymer chains intertwine and are relatively fixed in place by steric effects. These entanglement effects begin to occur at low chain length and have rather significant effects on the mechanical properties of the bulk polymer. Even in melt form, due to increasing entanglement, the viscosity of a polymer is related to the cube of the molecular weight. At longer chain lengths (>100 monomers) this entanglement effect is often the dominant crosslinking effect, especially at only small variations in degree of chemical crosslinking.

Gel modulus can also be affected by the polymer concentration in solution prior to crosslinking. Gelation procedures typically dictate that gels form from specific volume of solution, usually to obtain a particular thickness of shape of the gel. Under this condition, changing the concentration of polymer in the solution results in a change in total amount of polymer in the network while the volume of the total gel remains unchanged. Since solvent occupies the volume of gel not occupied by polymer, decreasing the polymer amount effectively increases the volume of solvent. This loss of connectivity allows the structure to yield more under applied stresses, meaning a lower modulus for those structures.

Swollen and Dehydrated Hydrogel Coating

One potential problem with the use of a hydrogel coating is maintaining its integrity and adherence to the neural implant during the process of implanting the neural implant into the body, i.e., into the brain tissue. Weak adhesion between the hydrogel and the neural implant potentially can damage the hydrogel during implantation. This potential problem can be overcome by providing the hydrogel coating in a dehydrated state, whereupon following implantation, the dehydrated hydrogel can be rehydrated from the high water content in and surrounding the brain tissue, returning the hydrogel coating to an operable, swollen configuration. The dehydrated hydrogel coating has the mechanical rigidity to remain adhered to the neural implant during the implantation process.

Polyethylene glycol hydrogels of various formulations were fabricated and produced elastic moduli ranging from 13 kPa to 687 kPa, which lie within two orders of magnitude of the elastic moduli of the brain (≈5 to 6 kPa). The swelling process in the brain phantom was observed to be slower than in unconstrained swelling. The equilibrium swollen hydrogel was also slightly smaller in the constrained state, implying the strain is being distributed between the hydrogel and the brain phantom.

Hydrogels are materials formed from a cross-linked network of polymer chains. A variety of hydrogels can be developed by manipulating the polymer composition and the cross linking mechanism. Crosslinks can be formed through both chemical and physical means. The result is a hydrophilic material with unique mechanical and physical properties, including the ability to imbibe water and swell. Many hydrogels have been developed that are environmentally sensitive to factors such as pH, temperature, and ionic strength. Hydrogels are frequently used in bioengineering because they are easily modified and highly biocompatible.

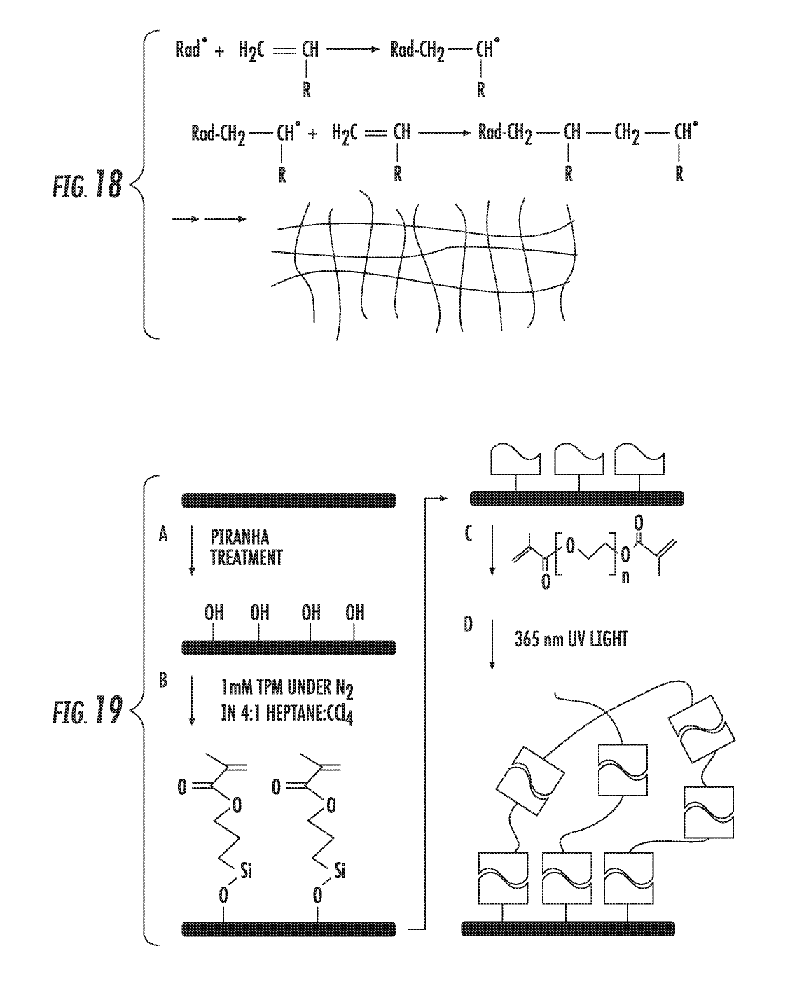

One frequently used mechanism for forming crosslinks is photo-polymerization. The process utilizes light and photo-initiators to induce free radical polymerization of molecules. This method is beneficial because of its fast curing time, spatial and temporal control, and ability to be conducted in ambient temperatures. This allows for the formation of hydrogels on complex shapes, such as coatings on surfaces. The photo-polymerization process is initiated when a photo-initiator molecule is exposed to a specific wavelength of light and forms a radical species. The photo-initiator can be mixed into the hydrogel precursor solution so that after activation, the radical species that are formed will cause the polymerization process to proceed throughout the bulk of the solution to form the hydrogel. FIG. 18 depicts the photo-polymerization process to generate free radicals to initiate the polymerization process, with the representation of the hydrogel being idealized.

Polyethylene glycol (PEG) is a synthetic polyether that is biologically compatible due to its low toxicity and hydrophillicity. It is frequently used in vivo because it does not activate an immune response and prevents protein adhesion to surfaces. As shown in Table 2 below, PEG chains can be easily functionalized with terminal acrylate groups to form PEG-diacrylate or PEG-dimethacrylate for crosslinking purposes.

TABLE 2
Structure of three forms of poly (ethylene glycol) polymers

| Name | Structure |
|---|---|
| Poly(ethylene glycol) | 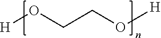 |
| Poly(ethylene glycol) diacrylate | 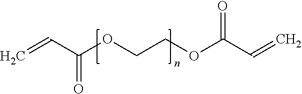 |
| Poly(ethylene glycol) dimethacrylate | 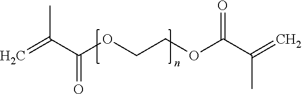 |

A useful property of hydrogels is their ability to swell when placed in a thermodynamically compatible solvent. The tendency of systems towards higher entropy states drives the thermodynamic force of mixing, which causes the hydrogel to expand. The elastic force from the stretching polymer chains keeps the hydrogel together. The hydrogel will continue to imbibe the solvent until the increase in elastic energy of the chains balances the decrease in free energy from mixing. No additional swelling will occur and the hydrogel will stay at equilibrium. This effect is described the Flory-Rehner equation which can be rearranged to give an estimate of the average molecular weight between crosslinks.

$$\frac{1}{\overline{{M}_{c}}} = \frac{2}{{M}_{n}} - \frac{( \frac{v}{{V}_{1}} ) ( ln ( 1 - {v}_{2 , s} ) + {v}_{2 , s} + χ * {v}_{2 , s}^{2}}{{v}_{2 , s}^{1 / 3} - \frac{{v}_{2 , s}}{2}} \qquad ( 1 )$$

where Mₙ is the number average molecular weight of the polymer chains, v is the specific volume of the bulk polymer in the amorphous state (0.893 cm3/g), V1 is the molar volume of the solvent (18 cm3/mole), X is the polymer-solvent interaction parameter, and v2,s is the polymer volume fraction in the swollen state.

The interaction parameter is relatively constant at x=0.426 at room temperature for polymer volume fractions ranging from 0.04 to 0.2. The polymer volume fraction in the swollen state is a simple ratio of the volume of polymer to the total gel volume. It can be expressed in terms of the mass ratio and the densities of the polymer and solvent.

$${v}_{2 , s} = \frac{{v}_{p}}{{v}_{g}} = {Q}_{v}^{- 1} = \frac{1 / {ρ}_{p}}{{Q}_{m} / {ρ}_{s} + 1 / {ρ}_{p}} \qquad ( 2 )$$

Qm is the mass ratio or the swelling ratio and is defined as the mass of the gel over the mass of the polymer. The polymer volume fraction measures how much fluid can be taken up and retained by the hydrogel while the average molecular weight Mc between crosslinks is a measure of the degree of crosslinking.

A modified equation for the average molecular weight between crosslinks is used for hydrogels prepared in the presence of water. This altered equation takes into account the water-induced elastic contributions to swelling.

$$\frac{1}{\overline{{M}_{c}^{*}}} = \frac{2}{{M}_{n}} - \frac{( \frac{v}{{V}_{1}} ) ( ln ( 1 - {v}_{2 , s} ) + {v}_{2 , s} + χ * {v}_{2 , s}^{2}}{{v}_{2 , r} [ {( \frac{{v}_{2 , s}}{{v}_{2 , r}} )}^{1 / 3} - ( \frac{1}{2} ) ( \frac{{v}_{2 , s}}{{v}_{2 , r}} ) ]} \qquad ( 3 )$$

The v2,r term is the volume fraction of the hydrogel in the relaxed state. This is the state of the hydrogel just after crosslinking but prior to being submerged in solvent to swell. The swelling ratio and the average molecular weight between crosslinks are the most useful values used to characterize hydrogel network structure.

An elastic modulus for the hydrogel can be estimated using the rubber elasticity theory. This theory can be applied because up to deformations of 20%, hydrogels behave elastically and are capable of returning to their initial dimensions. The stress to a hydrogel sample is

$$τ = \frac{ρ RT}{{M}_{c}^{*}} ( 1 - \frac{2 \overline{{M}_{c}}}{\overline{{M}_{n}}} ) ( α - \frac{1}{{α}^{2}} ) {( \frac{{v}_{2 , s}}{{v}_{2 , r}} )}^{1 / 3} \qquad ( 4 )$$

where α is extension parameter, or the final length over the initial length. This theory assumes Gaussian behavior of the polymer chains. The equation can be rearranged to solve for an approximation of the elastic modulus, which approaches a third of the Young's Modulus as the limit of a approaches 1.

$$\frac{τ}{( α - \frac{1}{{α}^{2}} )} = \frac{ρ RT}{{M}_{c}^{*}} ( 1 - \frac{2 \vec{{M}_{c}}}{\overline{{M}_{n}}} ) {( \frac{{v}_{2 , s}}{{v}_{2 , r}} )}^{1 / 3} \qquad ( 5 )$$

The disclosure can be further understood with reference to the following non-limiting examples.

### EXAMPLE 1

### Hydrogel Coating

The effects of poly(ethylene glycol) (PEG) hydrogel coatings for neural implants (such as glass brain implant devices) on strain fields imposed by those devices to brain tissue due to micromotion in the brain were studied. PEG hydrogels were created using macromers of 2000-8000 M_{w} and 5-20 wt. % in solution. The moduli of the hydrogels were calculated via Hertzian analysis of force-deflection curves produced using an atomic force microscope (AFM) tip as a nanoindenter. The moduli of the samples did not change significantly with change in macromer M_{w}, but did change with solution concentration. 20% gels had moduli of 120-180 kPa and 5-10% gels had moduli of 0-20 kPa. The strains imposed by the coated devices were lower at the surface by 30% as compared to uncoated and the strain field dropped off much more quickly.

Materials: Poly(ethylene glycol) (PEG) (Mw≈2000-8000 g/mol), methacrylic anhydride, 2-isocyanatoethyl methacrylate (IEM), ethyl ether, and triethylamine (TEA) were purchased from Sigma-Aldrich and used as received. Dichloromethane, sulfuric acid, hydrogen peroxide solution, 3-(Trichlorosilyl)propyl methacrylate (TPM), 2-hydroxy-4′-(2-hydroxyethoxy)-2-methyl propiophenone, 2,2-dimethoxy-2-phenylacetophenonc and carbon tetrachloride were purchased from Sigma-Aldrich. Heptane was purchased from Macron Fine Chemicals and used as received. The glass slides used were 12 mm diameter with a thickness of 0.16-0.19 mm and were purchased from Electron Microscopy Sciences. Glass capillaries were purchased from VWR. Fluorescent polystyrene particles with a diameter of 5.9 μm were purchased from Polysciences, Inc. and used as received.

Equipment: The Cure Spot 50 from ADAC systems controlled the ultraviolet radiation source during gelation. Strain field measurements were performed using a standard bright field optical microscope from Micro-Tech Optical, Inc. Force and modulus measurements were taken using an atomic force microscope from Veeco, the Nanoscope IV with Multimode and Picoforce.

PEG Synthesis: Poly(ethylene glycol) dimethacrylate was prepared. PEGDM was formed from the reaction of various PEGS, MA and IEM. An example of the synthesis of a 5 k PEGDM is as follows. PEG (5 g, ≈0.001 mol), 2.2 equiv of MA (0.34 g, 0.0022 mol), and TEA (0.2 mL) were reacted in 15 mL of dichloromethane over freshly activated molecular sieves (≈3 g) for 4 days at room temperature. The solution was filtered over alumina and precipitated into ethyl ether. The product was filtered and then dried in a vacuum oven overnight at room temperature.

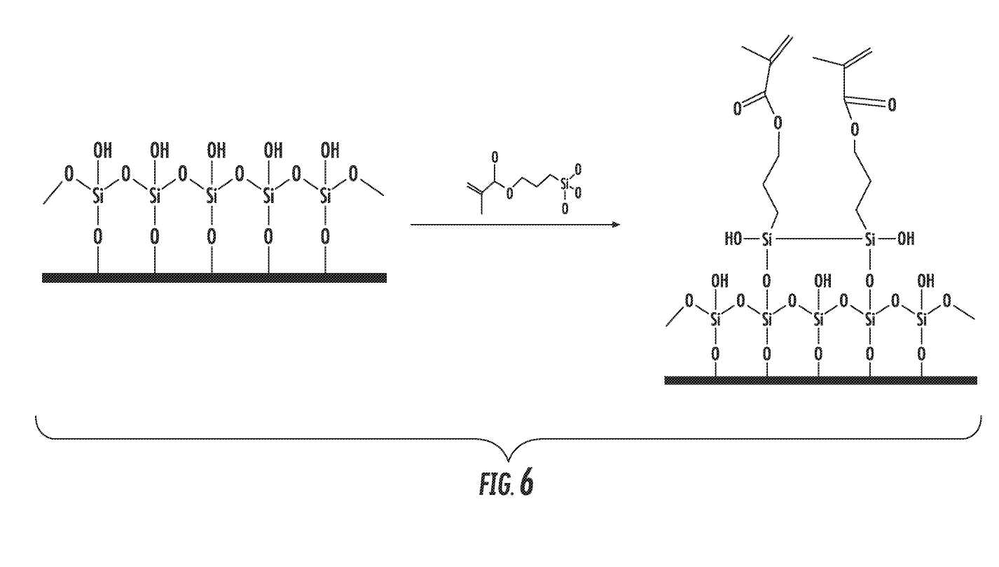

Surface Functionalization: Glass substrates were functionalized using standard protocols for surface modification. Substrates were cleaned in a 3:1 sulfuric acid:hydrogen peroxide “piranha” solution and then treated with TPM in a 1 mM solution of 4:1 heptane:carbon tetrachloride in a nitrogen atmosphere. The substrates were washed and dried after each step. This treatment forms a monolayer of methacrylate groups on the surface on the glass to provide points for the PEG to covalently bond to the surface to prevent delamination. FIG. 6 depicts surface functionalization of glass substrates. In particular, FIG. 6 depicts a clean glass surface getting functionalized to have methacrylate groups for PEG to bind to by TPM in 4:1 Heptane:CCl₄ environment.

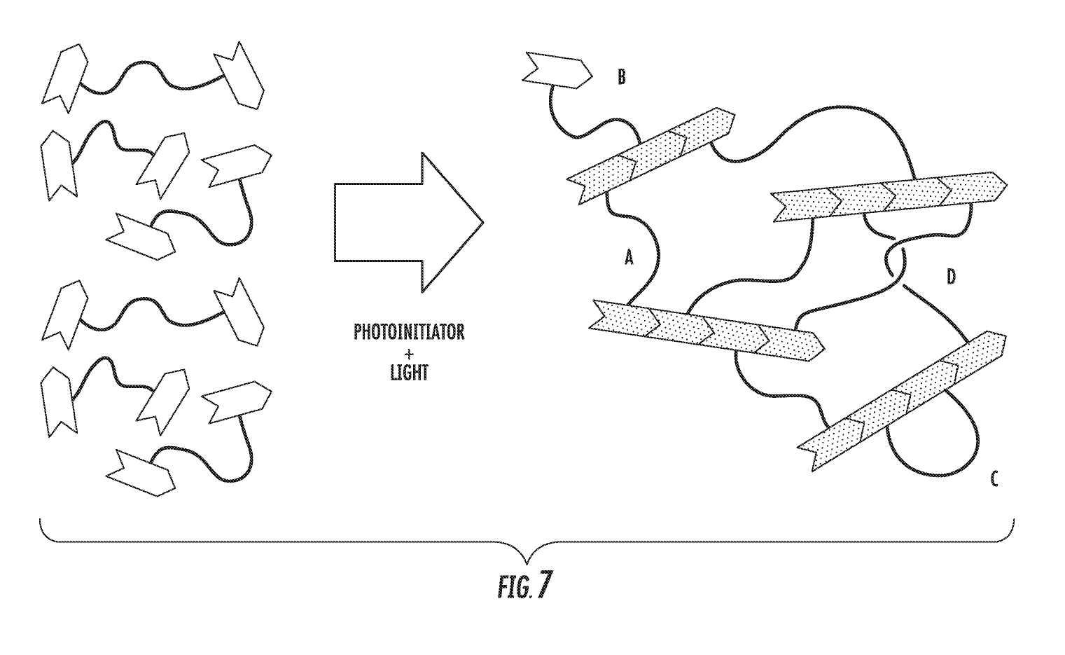

Gel Formation: Functionalized glass substrates were then coated with a solution containing dissolved PEG macromers at the concentrations of 5, 10, and 20% by weight, with 0.5% photoinitiator. Samples were coated via either a tube filling process, for capillaries, or direct pipetting onto the surface. The solutions were exposed to 365 nm ultraviolet radiation to crosslink the PEG via free radical polymerization. The solution was exposed to UV for 90 seconds or, in the case of low concentrations, until gelation occurred for direct pipetting. The glass capillaries were suspended inside a larger glass tube for tube filling and solution was dripped into the tube and then pulled across the surface of the capillary through capillary action. This method was utilized to ensure a uniform thickness of the hydrogel coating across and around the device. FIG. 7 depicts a schematic representation of the PEGDM network showing (a) crosslinked PEG chains and network defects, including (b) unreacted acrylate terminuses, (c) PEG cycles and (d) change entanglements. The shaded arrow shapes in FIG. 7 represent reacted acrylates, the unshaded arrow shapes represent unreacted acrylates, and dark lines represent PEG chains esterified to the acrylic acid. For clarity, short acrylate chains are shown, but in actual gels, these chain lengths may be much longer.

Force Measurements and Modulus Calculations: The atomic force microscope (AFM) is well suited for probing the local elasticity of small very soft samples when the cantilever tip is used as a nanoindicater. Samples with swollen PEG hydrogel were placed in the AFM and probed by a spherical polystyrene tip with a diameter of 45 μm and a cantilever spring constant of 14N/m. Tip deflection as a function of depth of indentation was measured. The Young's moduli of the gels were then determined via analysis Hertzian analysis of the resulting force-displacement curves. This analysis assumes that the hydrogels are deforming in a linear elastic fashion and there is negligible adhesion between the gels and the AFM tip. The AFM was used to measure the relationship between tip deflection and depth of indentation into the gel. The force applied by the tip was calculated by

F=k_{c}(d−d₀) (6)

where k_{c} is the spring constant of the cantilever, d is the measured deflection, and d₀ is the deflection offset at the point of contact. Using the spherical geometry of the tip it was then possible to derive the modulus from the relationship

$$F = {\frac{4 {ER}^{\frac{1}{2}}}{3 ( 1 - {v}^{2} )} [ ( z - {z}_{0} ) - ( d - {d}_{0} ) ]}^{3 / 2} \qquad ( 7 )$$

where z is the measured translation of the cantilever, z₀ is the translation of the cantilever at the contact point, R is the radius of the tip, v is the Poisson's ratio, which was assumed to be 0.5, and E is the Young's modulus.

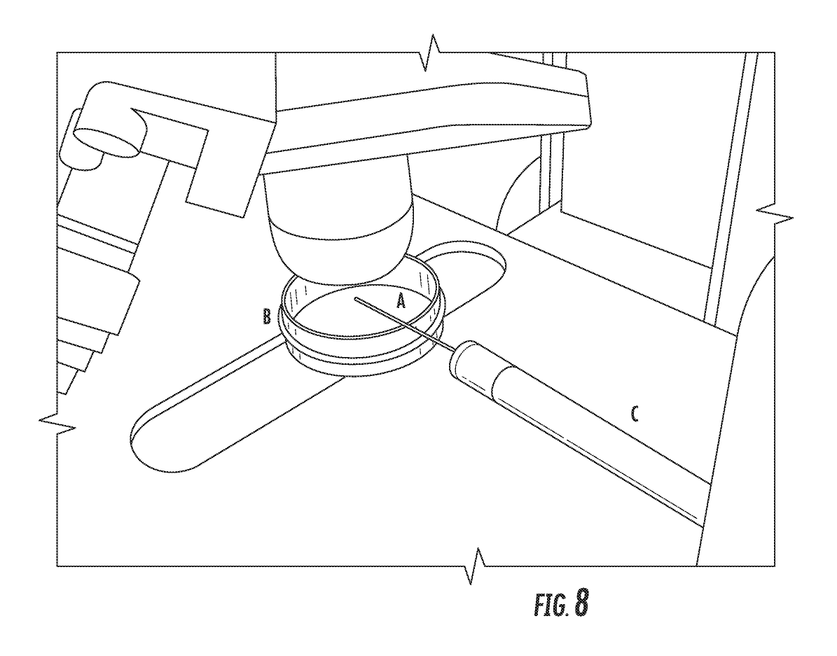

Particle Tracking Analysis: The brain undergoes micromotion in the radial directions (along device axis) due to vascular (1-3 μm) and respiratory pulsations (2-40 μm). Agarose gel with fluorescent particles suspended was formed around the coated device to prevent shearing during insertion. The device was then displaced 20-40 μm in the radial direction to simulate micromotion. FIG. 8 depicts a strain field measurement setup. The strain field measurement setup includes a coated glass capillary A, which is immersed in agarose gel in a petri dish B under optical microscope. It is held by a capillary holder C, which is connected to a stepper motor capable of simulating micromotion in the brain in the radial direction.

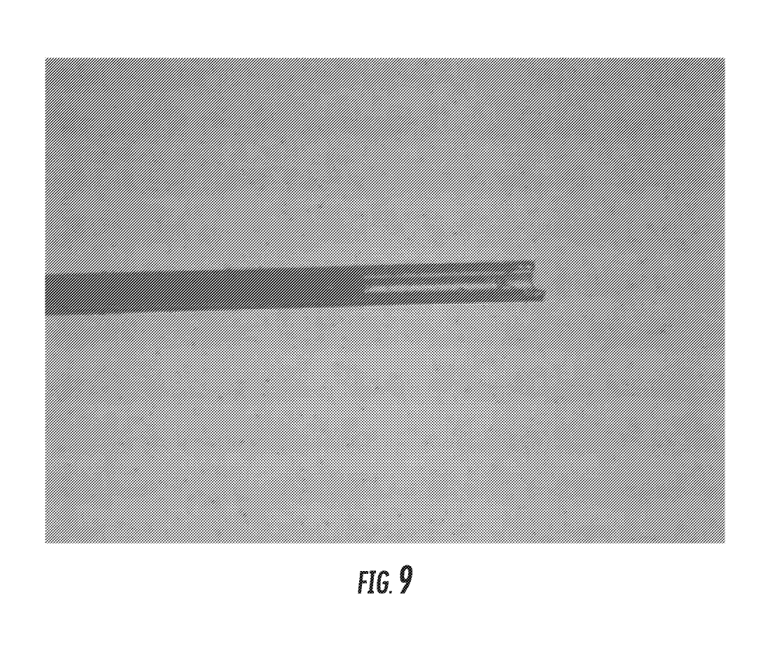

Bright field images before and after deformation were analyzed to obtain the resulting displacements for each particle. The strain fields in the gel were calculated from the observed particle displacements using the particle image velocimetry plugin for ImageJ. Strain fields imposed by different samples were compared by examination of the maximum particle displacement around the implant as well the variation displacement vectors as a function of distance from the implant. This analysis determined the effect that hydrogel coatings have on cells directly around the implant, as well as estimate the volume of brain tissue around the implant that experiences stress resulting from micromotion respectively. FIG. 9 depicts an optical bright field microscope image of an uncoated device. The black capillary is the uncoated glass device. The device is surrounded by agarose gel, and the multitudes of black spots are the polystyrene particles suspended in the gel used for strain field analysis.

Modulus Calculations of Gels: The moduli were visualized by plotting as a function of both molecular weight of the original chains of PEG and by the concentration of the polymer in the gel precursor solution.

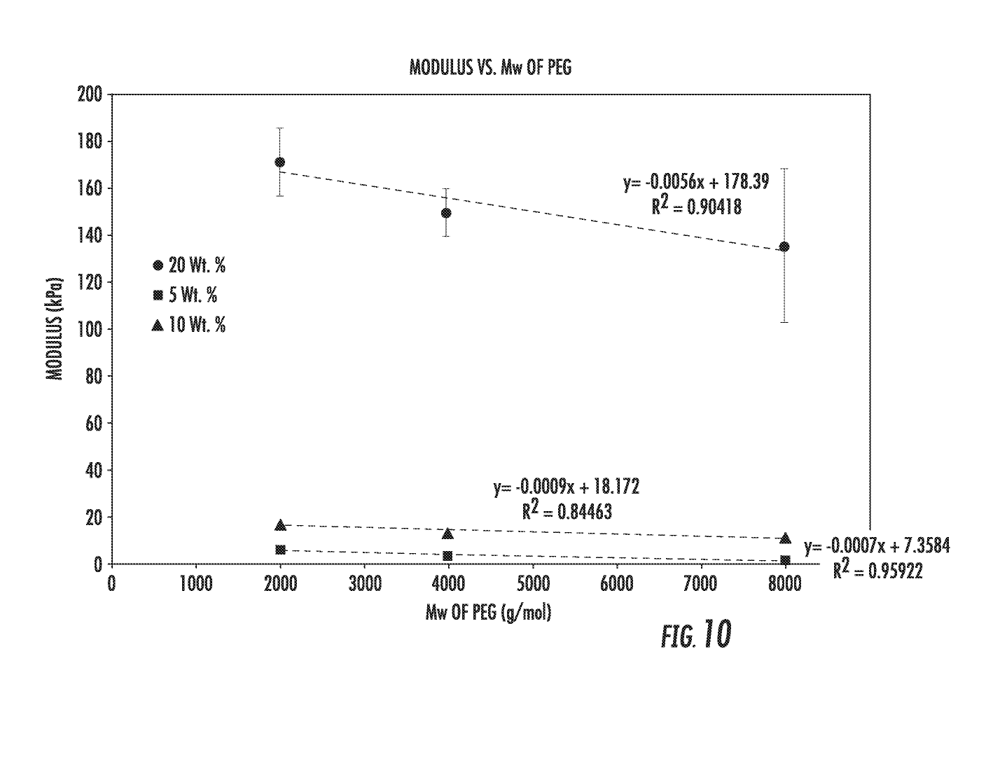

As depicted in FIG. 10, when comparing the moduli of the samples as a function of molecular weight of the original chains, varying the molecular weight of the polymer had little to no effect on the resulting modulus. Increasing the molecular weight increased the resulting physical crosslink density slightly, but greatly decreased the chemical crosslink density. A great decrease in chemical crosslink density, without any physical crosslinking would result in a great decrease in modulus. In these samples, however, the degree of physical crosslinking seemed to be the dominant factor in determining the modulus of the samples. The change in chemical crosslink density, modulated through the change in molecular weight, at least on this scale, had no significant effect on the modulus of samples of similar precursor solution concentration.

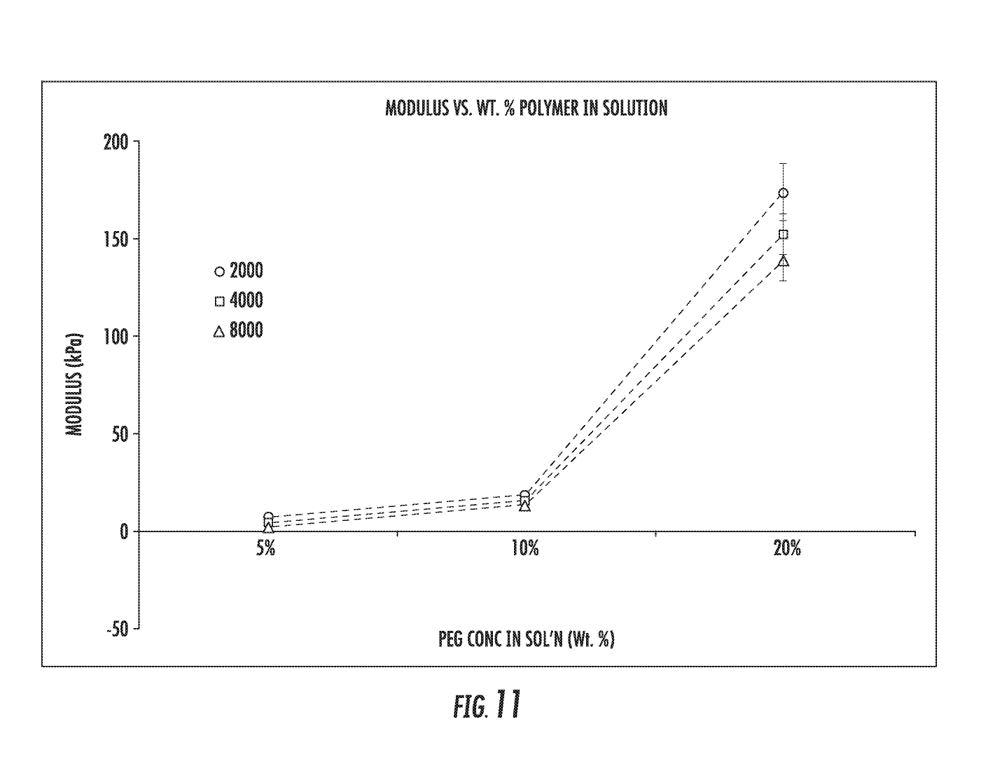

Changing the concentration of PEG in the precursor solution seemed to have a large effect on modulus of the samples. As depicted in FIG. 11, the modulus of the gel was observed to increase as a function of polymer concentration for all chain lengths. This suggests a greater crosslink density at higher polymer concentrations. The 10% gels had slightly higher moduli than the 5% gels, but the set of 20% gels had considerably higher moduli than the other gels. This result may be due to the lower concentration polymer being too disperse in solution to form a homogenous distribution of clusters. This resulted in large defects in matrix, and reduced crosslinking, and thus a reduced modulus. At low concentration, it also was more likely that the reactive terminuses of a single PEG chain react with themselves, further reducing the connectivity of the resulting gel. Somewhere in the range between 10-20% a critical concentration is reached at which homogeneity is reached and the gel can properly form.

Strain Field Analysis: The particle displacement vector fields were visualized by determining the movement of each polystyrene bead in the gel near the device tip after simulating micromotion through movement of the device and then plotting that movement as a vector. Cross sections of the vector plots were taken near the device tips and graphed showing the change in vector magnitude as a function of distance from the device.

The strain imposed on the gel was highest right near the devices and decreased with distance from the device on all sides. Both the particle displacement at the surface of the device and the displacement at longer distances were smaller when the devices had a PEG coating as compared to the control.

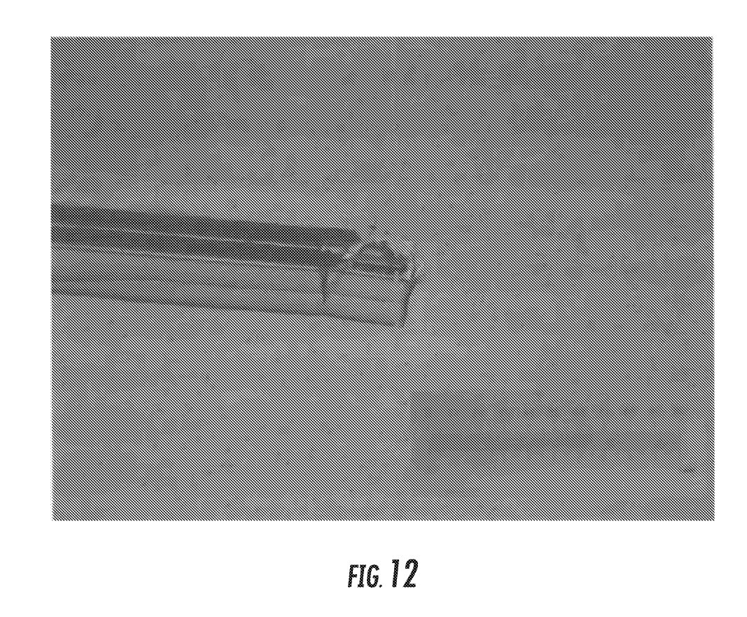

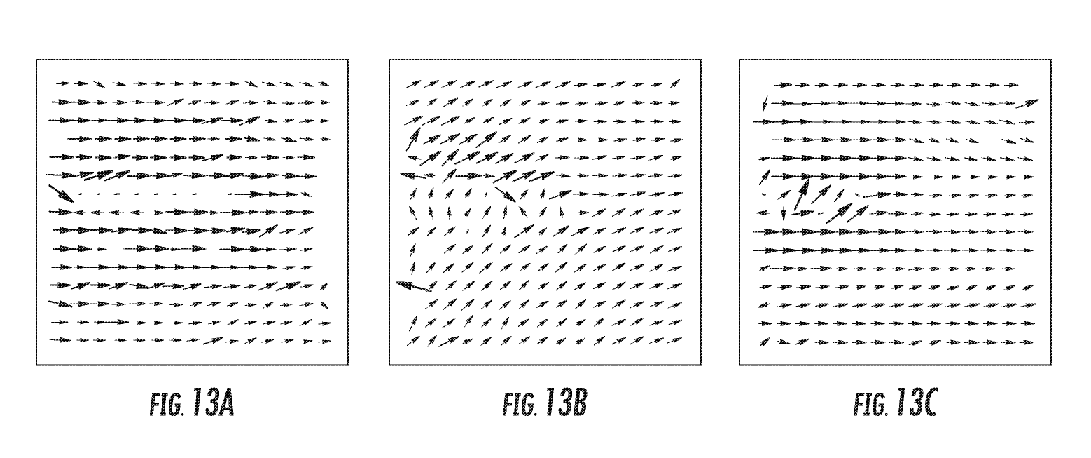

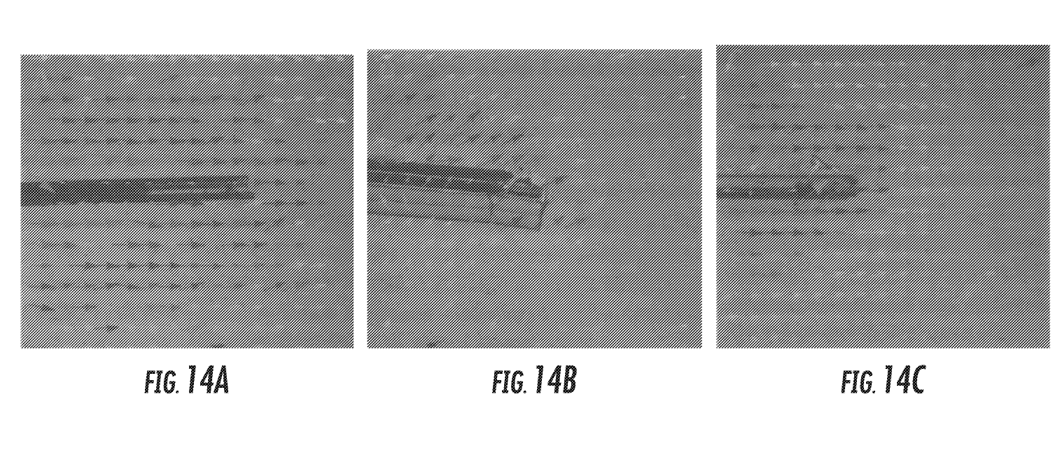

The polystyrene beads near the surface of the device in the uncoated sample move about 30 μm, which is the amount of movement applied to the device to simulate the micromotion. Near the surface of the hydrogel coated devices, which have a reduced modulus, the beads were found to move 15-20 μm. The displacements in the coated device samples also have a higher rate of decrease as a function of distance from the device than the uncoated devices. The exception to this was the coated sample made from PEG-2000. The particle displacement on one side of the device was comparable to the particle displacement of the uncoated, while the other side has displacement more similar to the coated device, as depicted in FIG. 12. This can be understood by looking at a picture of the device. The device has a hydrogel coating on one side, while the other side is essentially uncoated. For example, the black part in FIG. 12 is the glass capillary and the area below it is the PEG coating. There was very little coating above the device, leading to strain behavior in that region more similar to that of uncoated devices. The dashed line denotes the line on which the cross section of the displacement field was taken. This further corroborates the difference between imposed displacement fields from uncoated and coated devices. These finding also support those of other simulations that soft materials imposed lower strain on brain tissue undergoing micromotion than materials with high moduli. For example, FIGS. 13A-13C depict vector plots of particle displacement fields imposed by a device under about 30 μm motion on the surrounding agarose, with FIG. 13A depicting an uncoated device, FIG. 13B depicting PEG-2000 coating, and FIG. 13C depicting PEG-8000 coating. FIGS. 14A-14C depicts particle displacement field vector plot overlays that correspond to FIGS. 13A-13C.

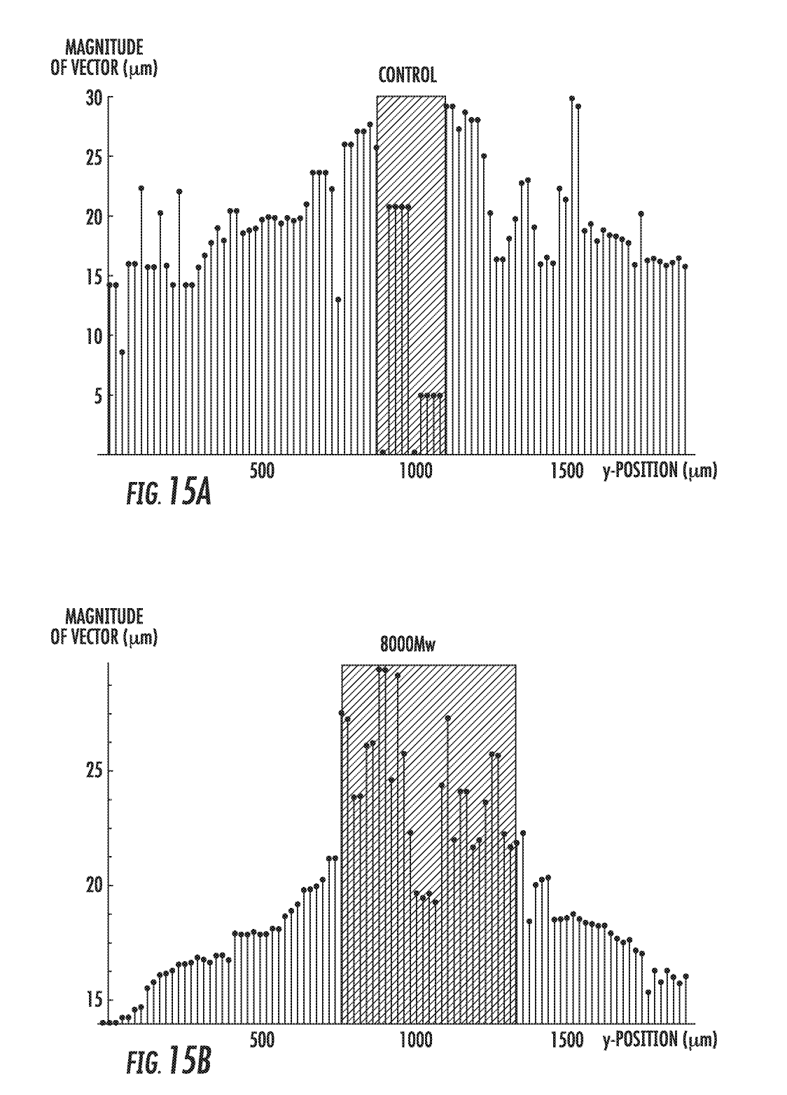

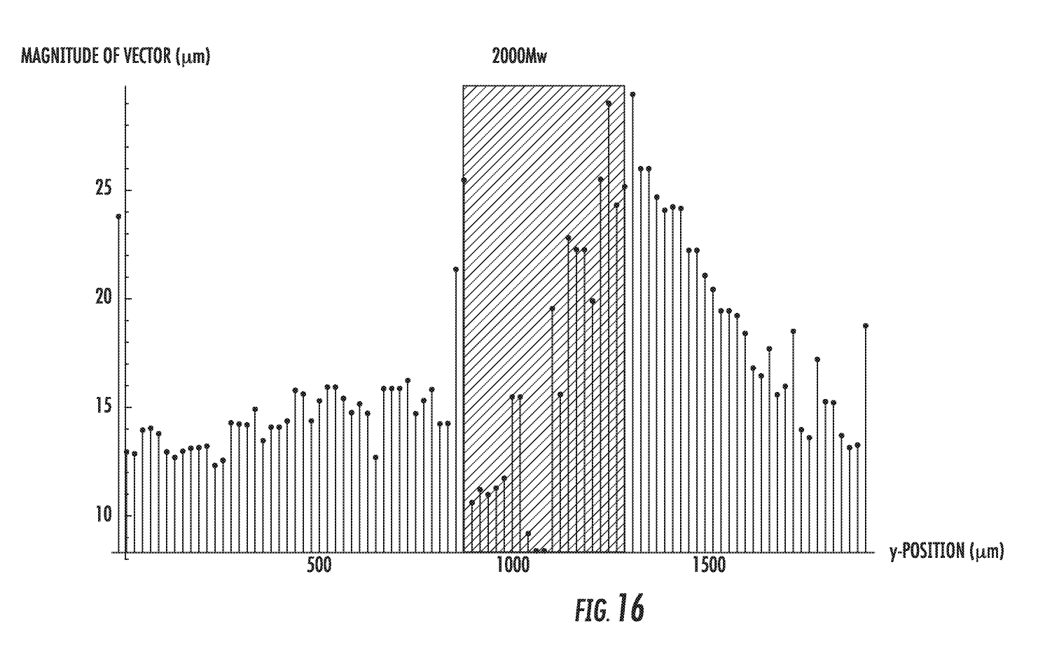

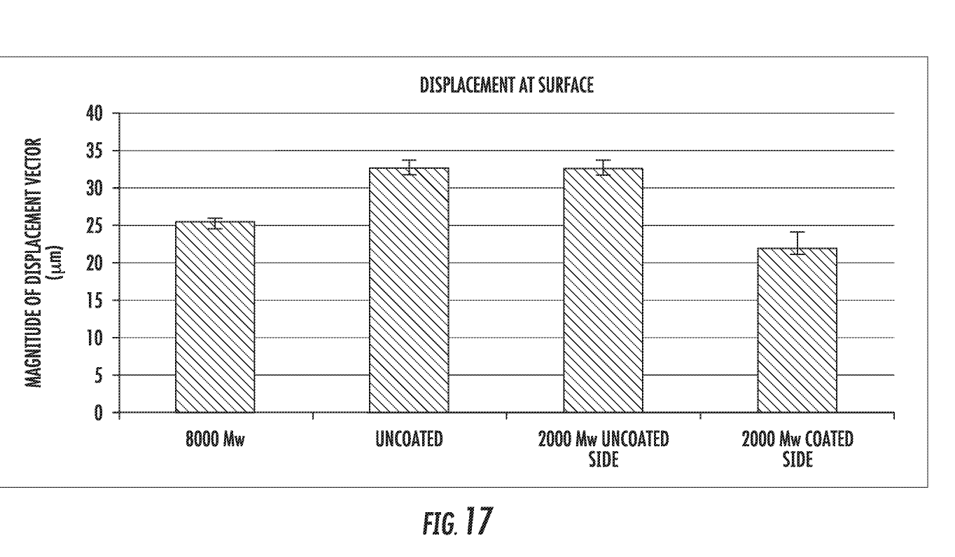

FIGS. 15A and 15B depict particle displacement field cross sections, with FIG. 15A showing the uncoated device and FIG. 15B showing the coated device (PEG-8000). The magnitude of the particle displacement vector is plotted. The shaded area is the actual device. The coated device is much larger because of the coating. The particle displacements are lower for the PEG-8000 coated device due to the lower modulus of the sample. FIG. 16 depicts a particle displacement field cross section of PEG-2000 device. The coated device only has a significant amount of coating on the bottom (corresponding to the left side) and the agarose on that side has a reduced particle displacement when compared to the nearly uncoated top (corresponding to the right side). FIG. 17 depicts displacement at the surface of various coated and uncoated devices. For the PEG-2000 coating, the two sides of the device were analyzed separately due to the loss of hydrogel coating on one side.

Conclusion: The use of PEG hydrogel coatings on glass brain implant devices reduces the strain field imposed by those devices on tissue due to brain micromotion. The lower modulus of the hydrogel coating acts as a remedy to the mechanical mismatch between the high modulus glass (≈69 GPa) and the low modulus brain tissue (≈5 kPa).

The moduli of these coating can be controlled to an extent using varying concentration of PEG in solution before gelation. It is possible that the modulus can also be controlled by varying the degree of swelling, or by varying the chain lengths to a much higher degree. Thickness of the gel may also be a factor in the moduli of the gel if the gels are thin enough that there are still residual effects of the high modulus glass even at the surface of the gel.

The reduction in strain field reduces the extent of glial scarring near the implant area of the device. In effect, the addition of PEG hydrogel coatings is expected to improve the effectiveness and longevity of the device.

The presence of the hydrogel coatings described herein significantly reduced the local strain surrounding the implant following micromotion. The strain imposed on the gel was highest near the devices and decreased with distance from the device on all sides. Both the strain at the surface of the device and the strain at longer distances were reduced when the devices had a PEG coating as compared to the control. The strains in the coated devices also have a higher rate of decrease as a function of distance from the device than the uncoated devices. The exception to this was the coated sample made from PEG-2000. The strain on one side of the device was comparable to the strain of the uncoated, while the other side has strain more similar to the coated device. This observation was due to the device having a hydrogel coating on one side, while the other side is essentially uncoated. This further support the difference between imposed strain fields from uncoated and coated devices.

### EXAMPLE 2

### Hydrogel Swelling

Materials: Poly(ethylene glycol) diacrylate (PEG-DA) with a molecular weight of 700 g/mole was obtained from Sigma-Aldrich (St. Louis, Mo.). Poly(ethylene glycol) dimethacrylate (PEG-DM) with molecular weights of 2000, 4000, 6000 and 8000 g/mole were synthesized using a published protocol. Fluorescent and non-fluorescent Polybead® Polystyrene microparticles of 6.0 μm were obtained from Polysciences, Inc. (Warrington, Pa.).

Preparation of Hydrogel: The hydrogel precursor solution was produced from a mixture of either form of PEG, deionized (DI) water and photo-initiator. The PEG was dissolved in DI water to form concentrations of 5, 10, and 20% m/v PEG. The photo-initiator, 2-Hydroxy-4′-(2-hydroxyethoxy)-2-methylpropiophenone (224 g/mole) was added (0.2% m/v) and the solution was mixed until the solids were fully dissolved.

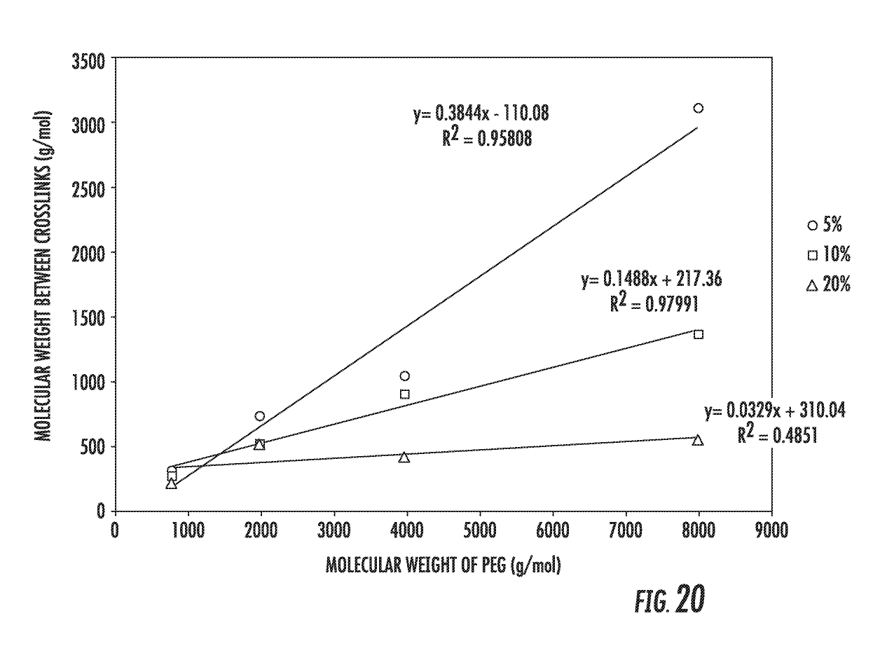

Preparation of Hydrogel Coating on Capillary: The production of a hydrogel coating on the glass capillary tube (a suitable proxy for a neural implant) was a two-step process performed at room temperature. First, a borosilicate glass capillary tube was functionalized with methacrylate groups to allow for stronger covalent bonds between the hydrogel and the tube. Then the tube was coated with a hydrogel precursor solution and exposed to UV light to form the hydrogel. The process is depicted in FIG. 19. The capillary tubes were treated with piranha solution to hydroxylate the surface. The capillary tubes were then functionalized with methacrylate groups using 3-(trichlorosilyl) propyl methacrylate (TPM). PEG-DM polymer chains were then introduced. Exposure to UV light startsed a photo-polymerization process that allowed the methacrylate end groups of the polymer chains to bond to the surface and crosslink with each other.

Capillary tubes with outer diameters of 150 μm were first treated with a piranha solution to remove organic residues and hydroxylate the surface. The solution was created from a 3:1 mixture of 80% sulfuric acid to 30% hydrogen peroxide. The tubes were submerged in the solution for 10 minutes, washed with DI water, and dried with nitrogen gas.

The tubes were then treated with 1 mM TPM in order to add methacrylate end groups. The tubes were added to a 4:1 ratio of heptane and carbon tetrachloride. TPM was introduced under nitrogen at 0.1644 μl/mL of the total solution. The solution was allowed to stir for 10 minutes. The tubes were washed with heptane, acetone, and DI water.

A glass capillary mold with an inner diameter of 400 μm was used to constrain the hydrogel coating over the capillary tube. The hydrogel precursor solution then filled into the empty space through capillary action. The mold was exposed to 365 nm UV light for approximately 30 s for each centimeter in length. Coated capillary tubes were stored in DI water at room temperature until use.

Gel Dependence on PEG MW and % PEG: Hydrogel precursor solutions of varying concentrations and molecular weights were synthesized as discussed above. 0.2 g of each solution was added to a 1.5 mL eppendorf tube. The tube was exposed uniformly to UV light for 90 s or until the solution gelled. The post-gelation weight was recorded and a hole was poked on the top of the eppendorf tube. The tubes were dried in vacuum for 2 days. The mass of the PEG (Mp), mass of the water (Mw), the relaxed gel mass (Mr) and the dry gel mass (Md) for each sample was measured. The dry gel mass was assumed to be equivalent to the polymer mass (Mp). These measurements were used to calculate the mass swelling ratios, the polymer volume fraction, the average molecular weight between crosslinks and the elastic modulus using equations (1)-(3).

Time Dependence of Swelling: Hydrogel coated capillaries were studied under an inverted optical microscope at different swelling states to determine the time evolution of swelling under free and constrained conditions. The capillary re-swelled in DI water in the unconstrained experiments. Constrained swelling experiments were conducted using a brain phantom composed of a 0.6% agarose gel with 0.005% w/v of Polybead@polystyrene 6.0 μm microparticles. Small 1 mm holes were drilled into the sides of 12-well cell culture plates and the agarose solution was gelled inside the wells. Hydrogel coated capillaries were immersed in DI water and allowed to swell to an equilibrium diameter (Ds). The capillary was dehydrated in a vacuum for 20 minutes and the diameter recorded (Dd). The dried capillary was then inserted into the agarose through the hole in the well plate as horizontally as possible to stay within the focus of the microscope. Images were taken at various time points. These images were used to determine an equation to describe the time dependence of swelling. Additionally, using ImageJ's Particle Image Velocimetry plug-in, the movement of the particles in the agarose solution was observed to generate a vector plot to show agarose and gel movement during different time intervals.

Gel Dependence on PEG MW and % PEG: Four different molecular weights and three different mass percentages of PEG were studied. The swelling parameters as shown in the table 3 below are the average of three determinations of the specimens. Some insolubility at the highest molecular weights (8000 g/mole) was observed. The hydrogel was difficult to cross-link at the lower mass percentages, and the gel was observed to be less ‘gel-like’ and more fluid.

TABLE 3
Summary of swelling parameters for different hydrogel formulations

| MW | m/v % | Qₘ | ν_{2,s} | M_{c}* |
|---|---|---|---|---|
| 700 PEG-DA | 5% | 20.2 ± 2.7 | 0.043 ± 0.004 | 306 ± 8 |
| | 10% | 10.5 ± 2.6 | 0.079 ± 0.008 | 269 ± 16 |
| | 20% | 5.9 ± 0.6 | 0.132 ± 0.015 | 221 ± 26 |
| 2000 PEG-DM | 5% | 20.7 ± 4.1 | 0.041 ± 0.002 | 733 ± 28 |
| | 10% | 10.1 ± 1.4 | 0.082 ± 0.011 | 514 ± 90 |
| | 20% | 7.6 ± 0.9 | 0.105 ± 0.002 | 523 ± 14 |
| 4000 PEG-DM | 5% | 18.9 ± 10.8 | 0.446 ± 0.005 | 1047 ± 150 |
| | 10% | 12.2 ± 1.6 | 0.069 ± 0.009 | 909 ± 163 |
| | 20% | 5.9 ± 0.3 | 0.132 ± 0.002 | 425 ± 36 |
| 8000 PEG-DM | 5% | 46.7 ± 10.8 | 0.020 ± 0.005 | 3124 ± 353 |
| | 10% | 13.3 ± 1.5 | 0.063 ± 0.003 | 1365 ± 109 |
| | 20% | 6.0 ± 0.1 | 0.129 ± 0.004 | 554 ± 36 |

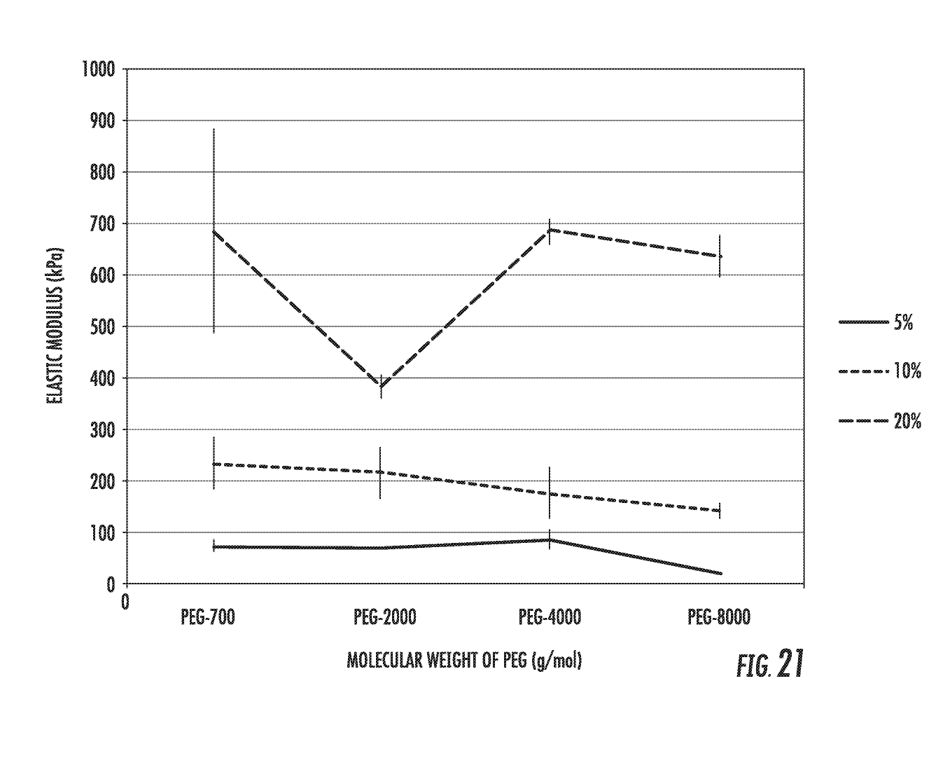

The swelling ratios are approximately the inverse of the mass percentages. The values for average molecular weight between crosslinks are shown in FIG. 20. Two trends were observed. First, an increase in polymer concentration led to a decrease in the average molecular weight between crosslinks, which suggests an increase in crosslink density. Second, the average molecular weight between crosslinks increased linearly with the molecular weight of the original polymer. This relationship is less clear in hydrogels with higher percentages of PEG due to increased numbers of physical crosslinks. This form of crosslinks include weak van der Waals' forces and entanglements, both of which are more significant at higher concentrations of polymer.

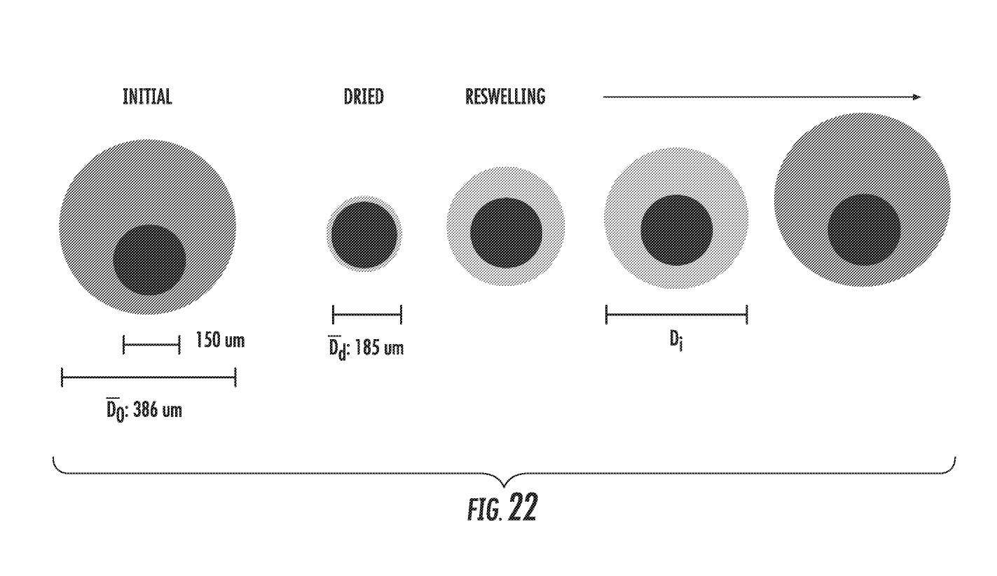

The elastic modulus for different hydrogels was calculated using equation (4). The unadjusted molecular weight between crosslinks values were used, as depicted in FIG. 21. The crosslink density decreases when the molecular weight between crosslinks increases, which leads to softer hydrogel with a smaller elastic modulus. The modulus of the 20% PEG-2000 gel is lower than expected and the modulus of the 5% PEG-4000 gel is higher than expected.

Swelling Ratio of Hydrogel-Coated Capillaries: A high degree of variability in the quality of the hydrogels was observed due to the difficulty in removing the hydrogel from the mold during the fabrication process. Part of the hydrogel coating often sheared off. Care was taken to use hydrogel-coated capillaries with no prominent defects for subsequent experiments. The diameter of the hydrogel-coated capillary was measured at different time points using ImageJ's ‘Distance Between Polylines’ plug-in to determine the swelling ratios from swelling and drying capillaries.

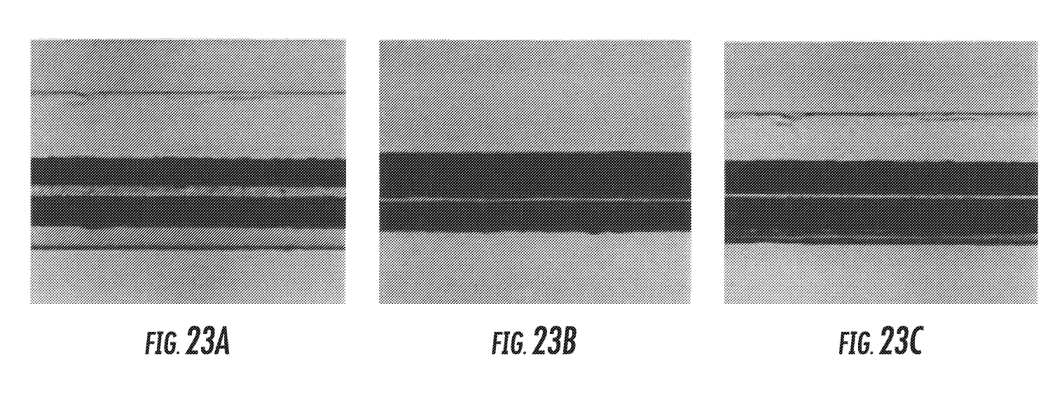

The cross-sectional area (Ai) of the hydrogel at each time point was estimated using the total measured diameter (Di) from ImageJ and the expected diameter of the capillary (Dc), which for these experiments were 150 μm. As depicted in FIG. 22, the capillary is not centered with the hydrogel after the fabrication process. The overall cross-section of the hydrogel is still assumed to be spherical due to the shaped of the mold. During re-swelling, the side with the thinner layer of hydrogel reaches equilibrium first.

$${A}_{i} = π [ {( \frac{{D}_{i}}{2} )}^{2} - {( \frac{150 µm}{2} )}^{2} ] \qquad ( 8 )$$

Once the cross-sectional area was determined, the swelling ratio was estimated using

$${Q}_{m} = \frac{( {A}_{t} - {A}_{d} ) {ρ}_{w} + {A}_{d} * {ρ}_{p}}{{A}_{d} * {ρ}_{p}} \qquad ( 9 )$$

where Aₜ is the cross-sectional area of a swollen gel at time t and Ad is the cross-sectional area of the dried gel. Aₜ is equal to Ao for calculations of the initial swelling ratio, where Ao is the initial diameter prior to drying out.

TABLE 4
Average parameters for hydrogel coated capillary
(10%, 700 MW PEG-DA)

| | Sample 1 | Sample 2 | Sample 3 | Average |
|---|---|---|---|---|
| Initial Diameter* (μm) | 390 | 389 | 381 | 386 |
| Dry Diameter (μm) | 188 | 178 | 189 | 185a |
| Initial Area (μm²) | 101,512 | 100,992 | 96,109 | 99,352 |
| Dry Area (μm²) | 9,935 | 7,275 | 10,230 | 9,147 |
| Swelling Ratio | 9.2 | 12.4 | 9.4 | 10.4 |

*Diameter refers to the full diameter of the hydrogel-coated capillary

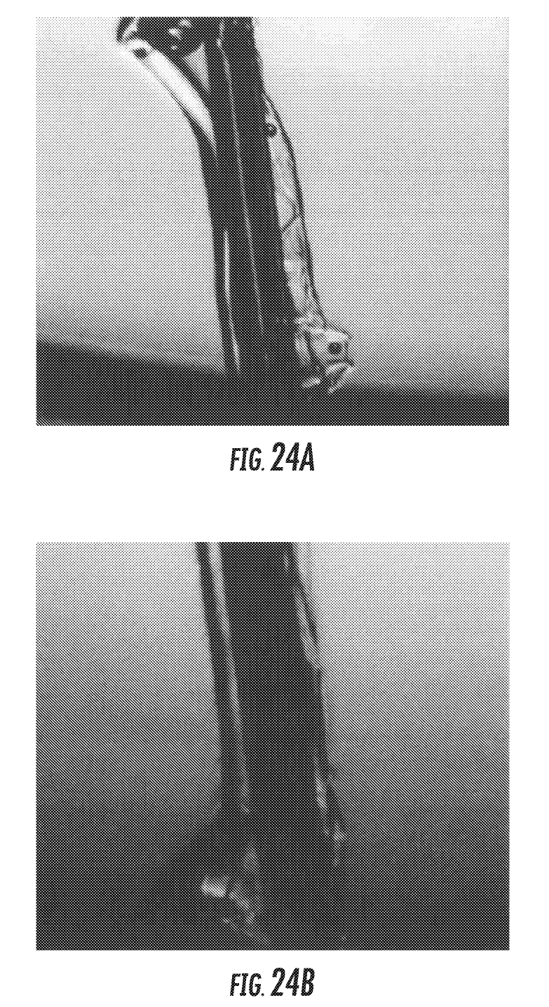

The swelling ratio of the hydrogel on the capillary (10.4) is within range of the swelling ratio of the hydrogel alone (10.5) as shown in table 3 and 4. To avoid confusion, the swelling ratio refers to the ratio of the initial gel mass to the dry gel mass. The re-swelling ratio refers to the ratio of gel mass after re-swelling to the dry gel mass. This suggests that having the hydrogel coated onto a capillary does not change the swelling parameters drastically. FIGS. 23A-23C depicts the various stages of the hydrogel. FIG. 23A depicts initial swollen hydrogel at equilibrium, FIG. 23B depicts dried hydrogel, and FIG. 23C depicts swollen gel after 1 minute of re-swelling.

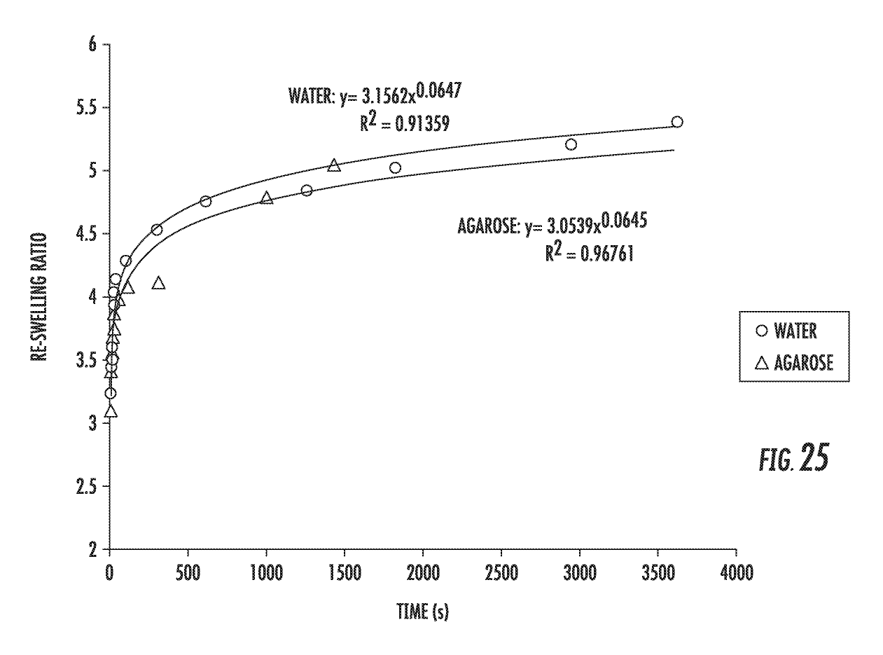

Time Dependence of Swelling: Insertion of a swollen hydrogel into the brain phantom caused shearing of the hydrogel, as depicted in FIGS. 24A and 24B. That is, when the hydrogel was inserted into the brain phantom without hydration, the hydrogel sears off and the coating is compromised. Dehydrating the hydrogel prior to insertion avoided this problem. Dehydrated hydrogels swelled after insertion into the brain phantom due to the agarose's high water content.

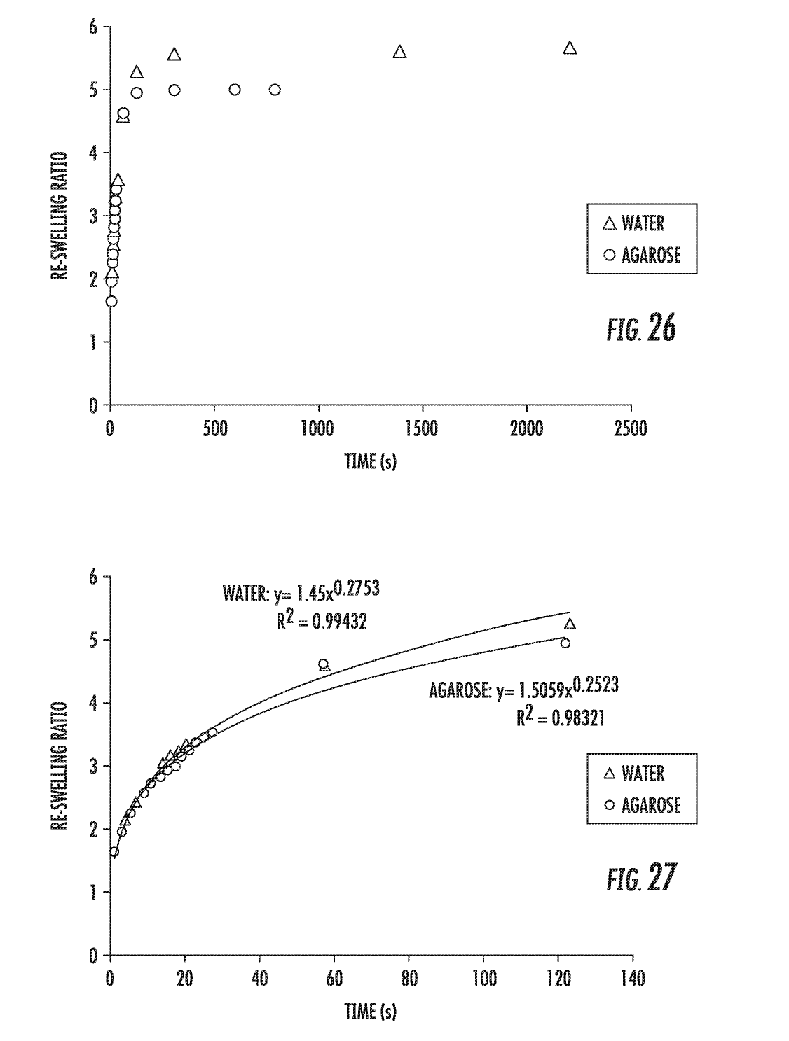

The corresponding hydrogel cross-sections were calculated using equation (8) and the re-swelling ratios using equation (9). The resulting time course of swelling was fit using a power law equation. FIG. 25 compares the time courses for swelling of a 10% MW hydrogel coated capillary in water and in agarose. The constrained swelling in agarose is slower than the unconstrained process in water as expected. The gel had not yet reached equilibrium at the time the last data.

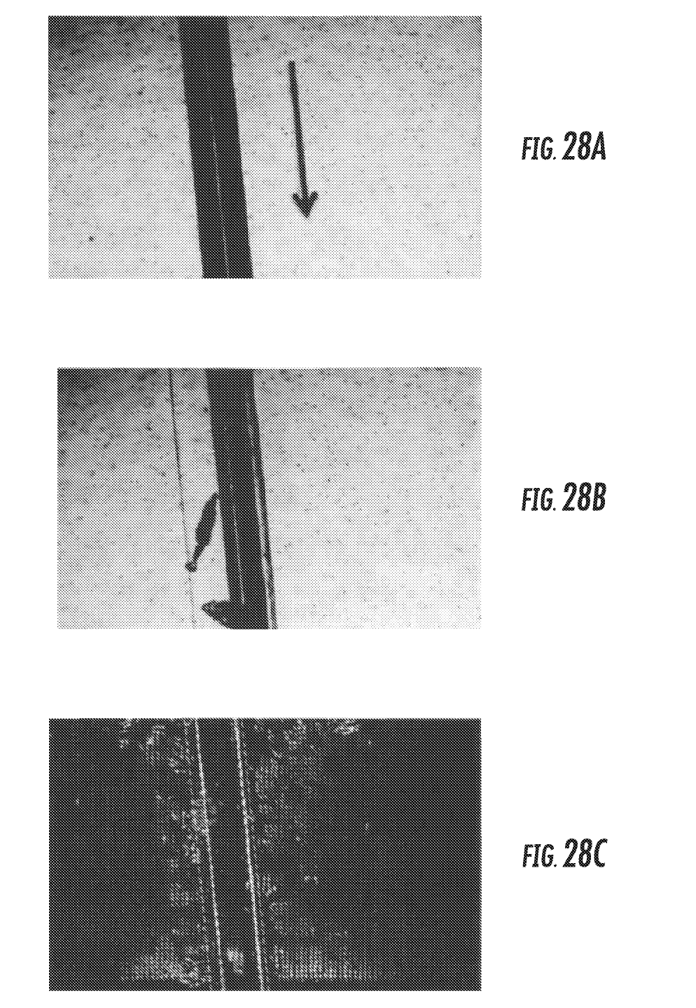

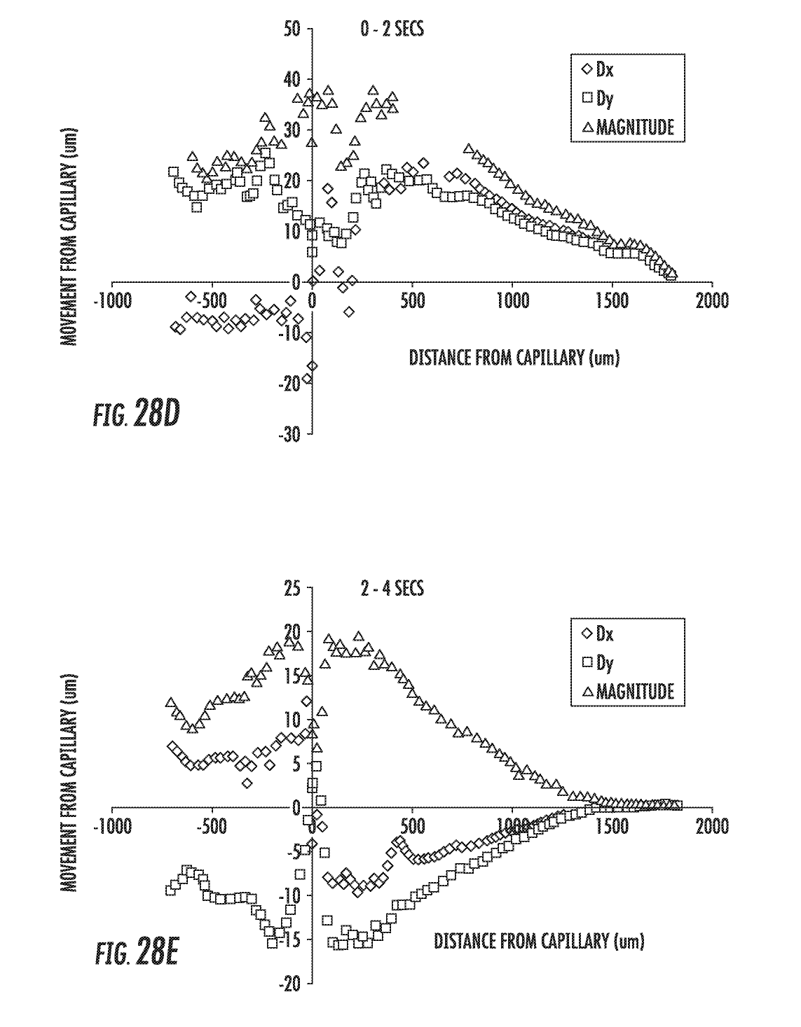

The time course experiment was repeated using 20%, 700 MW PEG-DA hydrogels. The re-swelling ratio levels off before 300 seconds, demonstrating that the hydrogel reached equilibrium, as depicted in FIG. 26, in both the water and agarose swelling experiments around 125 seconds. FIG. 27 shows the swelling time course before the gel reached equilibrium. The power law fit has an exponent of 0.25-0.27 which is higher than the exponent from FIG. 23A-23C for the 10% gel (0.065). This suggests that swelling in the 20% gel occurs more rapidly than in the 10% gel. FIG. 27 also confirms that swelling in agarose is slightly slower than the swelling in water.

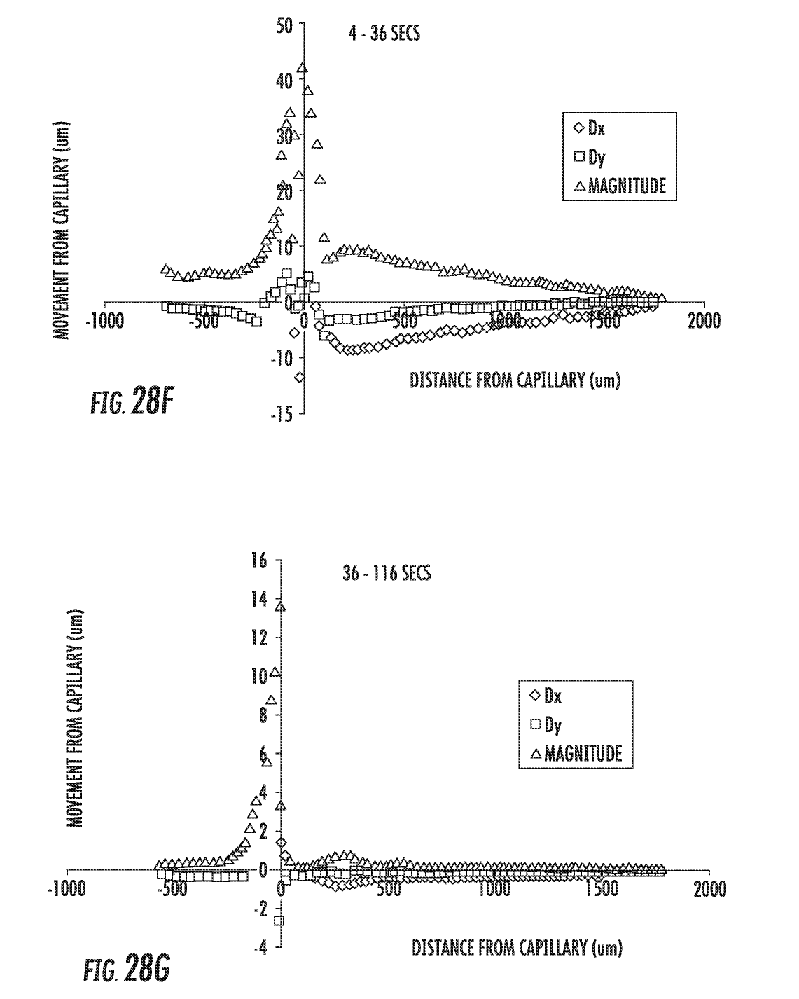

Vector Plot Observations: The effect of the hydrogel expansion on the agarose can be observed using Particle Image Velocimetry. The data presented in this section was for the gel shown in FIGS. 28A and 28B. FIG. 28A depicts the direction of implementation. FIG. 28B depicts the capillary after 300 seconds of swelling in agarose. FIG. 28C shows a sample vector plot for time interval 2-4 seconds with white lines indicating the capillary and gel boundaries that shows the direction and magnitude of the agarose movement. The average displacement in the x and y directions are plotted in FIGS. 28C-28H as a function of distance away from the capillary for different time intervals. The magnitude of the total displacement is also shown.

Positive x-displacement is defined as moving away from the capillary. Positive y-displacement is defined as moving down because the capillary was inserted in the downward direction. Negative x-axis values correspond to the agarose to the left of the capillary while the positive x-axis values correspond to the agarose on the right side.

The surrounding agarose moved in a similar direction as the capillary two seconds after implantation. The agarose shifted as if it was ‘pulled’ along, as indicated by the large displacements in the positive y-direction, as depicted in FIG. 28D, which also shows that the effects of implantation can be felt up to 2000 flm away. The agarose ‘readjusted’ and shifted upward to its undisturbed position as indicated by the large negative y displacements after 4 seconds (FIG. 28E). The displacements decrease linearly as the distance from the capillary increases in both FIGS. 28D and 28E. In FIG. 28F, the effect of both the agarose re-adjusting and the hydrogel expanding can be seen. The gel is still re-adjusting upwards although with smaller magnitudes than before on the right side of the capillary. The left side shows that the displacement is now decreasing exponentially with distance from the capillary, which is caused by the expansion of the hydrogel. FIG. 28G shows that after 36 seconds, the bulk of the agarose stopped moving and all displacement was due to the expanding hydrogel. Small perturbations seen on the graphs for the right side of the capillary is due the expanding hydrogel pushing the capillary to the right side slightly. Similar phenomena can be seen from 126-296 seconds in FIG. 28H.

Gel Dependence on PEG MW and % PEG: It was commonly reported in the literature that a recently cross-linked gel was allowed to swell in PBS for over 2 days to reach the equilibrium, swollen state. No additional swelling was detected in these experiments. The 10% and 20% gels experienced a loss in mass. This can likely be attributed to the small size of the hydrogel. The surface of the hydrogel began to dry out immediately upon exposure to open air, giving an inaccurate measure of the weight. The recently cross-linked state was considered to be the swollen state in calculations for v2,s to compensate. The relaxed state volume fraction (v2,r) was calculated using the initial masses of PEG and water. This adjustment produces an over-estimation in elastic moduli calculations since less swelling was detected than most likely existed in actuality.

Elastic moduli calculated using adjusted molecular weight between crosslinks values were approximately an order of magnitude larger than those shown in FIG. 21. Calculations using the unadjusted molecular weights produced elastic moduli values that were more consistent with those reported from previous literature. The coating may include compressive moduli of 34-360 kPa for 10-20% PEG-DM gels of MW 3400.

FIG. 21 did not show clearly than an increase in MW of PEG leads to a decrease in elastic modulus as expected. This is because the expected trend exists within the same order of magnitude. The error bars show that values within one order of magnitude cannot accurately be distinguished. This error can be attributed to the small sample sizes used. Masses as low as 0.009 g were measured. Values at this scale can be inaccurate so future hydrogel characterization experiments should use larger sample sizes. FIG. 21 did show that an increase in percentage of PEG leads to an increase in elastic modulus. Increasing the percentage of PEG from 5 to 20% was able to increase the elastic modulus by almost 2 orders of magnitude for the PEG-8000 gel.

Swelling Ratio of Hydrogel-Coated Capillaries: Drying out the hydrogel allowed the capillary to be inserted easily. The brain phantom provided the water necessary for the hydrogel to return to its initial swollen state. Most hydrogel coatings over the capillary were uneven, as shown in FIGS. 23A and 28A. An even hydrogel coating will expand radially and along the length of the capillary as well.

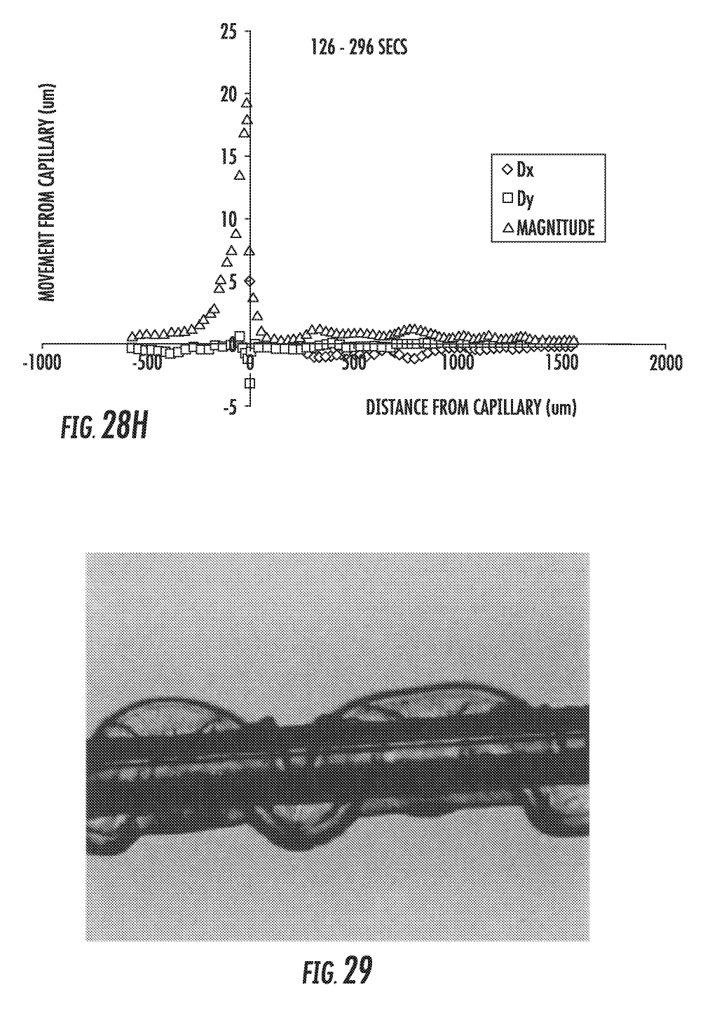

Buckling, as depicted in FIG. 29, was observed in some samples as the hydrogel swelled due to the hydrogel being chemically tethered down to the capillary. Samples where this occurred were not used because it would cause uneven strain on the surrounding agarose. This was more likely to occur in samples that were left to dry for over a day. That is, buckling of the hydrogel occurred during swelling in some samples when the hydrogel dried for extended periods of time.

Time Course of Hydrogel-Coated Capillaries: A power law fit assuming Fickian diffusion has the exponent of around 0.5. The fits for the swelling of 10% and 20% gels as shown in FIGS. 25 and 27 have exponents lower than 0.5. This “Less Fickian” behavior occurs when the water penetration rate is much lower than the polymer chain relaxation rate. Previous literature report power law fits only relevant for swelling below 60%. The power law fit was able to describe swelling up to equilibrium in these experiments. This is most likely because the total hydrogel thickness is very thin (on the scale of μm) while most literature reports have used hydrogels on the scale of millimeters.

The difference between swelling in agarose and water is very slight for both percentages of PEG, but both do show that swelling in agarose is slower. The equilibrium hydrogel state for the 20% gel in agarose was observed to be smaller than the equilibrium state of the gel in water. This implies that the hydrogel is slightly compressed as well, which matches hypotheses that the hydrogel will absorb some of the strain.

Experiments using the 20% hydrogels were able to reach equilibrium at around 2 minutes while the 10% hydrogels did not reach equilibrium within an hour. This can be explained by the difference in elastic moduli. FIG. 21 showed that the elastic modulus for the 20% hydrogel is higher than that of the 10% hydrogel. A higher elastic modulus allows the 20% hydrogel to push more against the agarose, expand faster and reach equilibrium sooner.

Swelling in the brain will be slower than swelling in the brain phantom. The brain phantom is composed of 99.4% water while the brain's water content is generally accepted to be around 75%. Water from the brain will be less accessible to the hydrogel, which will slow the swelling process. Mouse models will provide better insight into the swelling time course in brain tissue.

Vector Field Observations: Most of the strain from inserting the capillary occurred within the first four seconds as a result of the insertion. The surrounding agarose shifted in the direction of movement and then shifted backwards to readjust. The effects of this phenomenon could be felt up to 2000 μm away. The effects of the hydrogel expansion only affected the regions nearest to the capillary (within 250 μm).

One problem during the collection of the data was that in order to ensure that the inserted hydrogel was in focus, an initial portion of the capillary was inserted slightly. Any hydrogel on the capillary tip swelled immediately. This soft hydrogel tip caused less tearing of the agarose during implantation, but exacerbated the ‘pulling’ phenomenon, which resonated more throughout the gel.

Conclusion: PEG-DA and PEG-DM hydrogel coated capillaries were synthesized and characterized. The properties of the hydrogels were varied by altering the percentage and molecular weight of PEG. Calculated elastic moduli values ranged from 13 kPa to 687 kPa and are similar to that of the brain (5 kPa). Dehydration of the hydrogels allowed the capillary to be easily inserted into brain phantoms. Nearly instantaneously, the hydrogel began to imbibe water and swell. Hydrogels with higher moduli were observed to swell faster in both water and agarose. The 20% hydrogels reached equilibrium within 300 seconds while the 10% hydrogels did not reach equilibrium within an hour. Swelling in agarose was slower than swelling in water and reached a smaller equilibrium state.

### EXAMPLE 3

### Glial Scar Reduction in Vivo

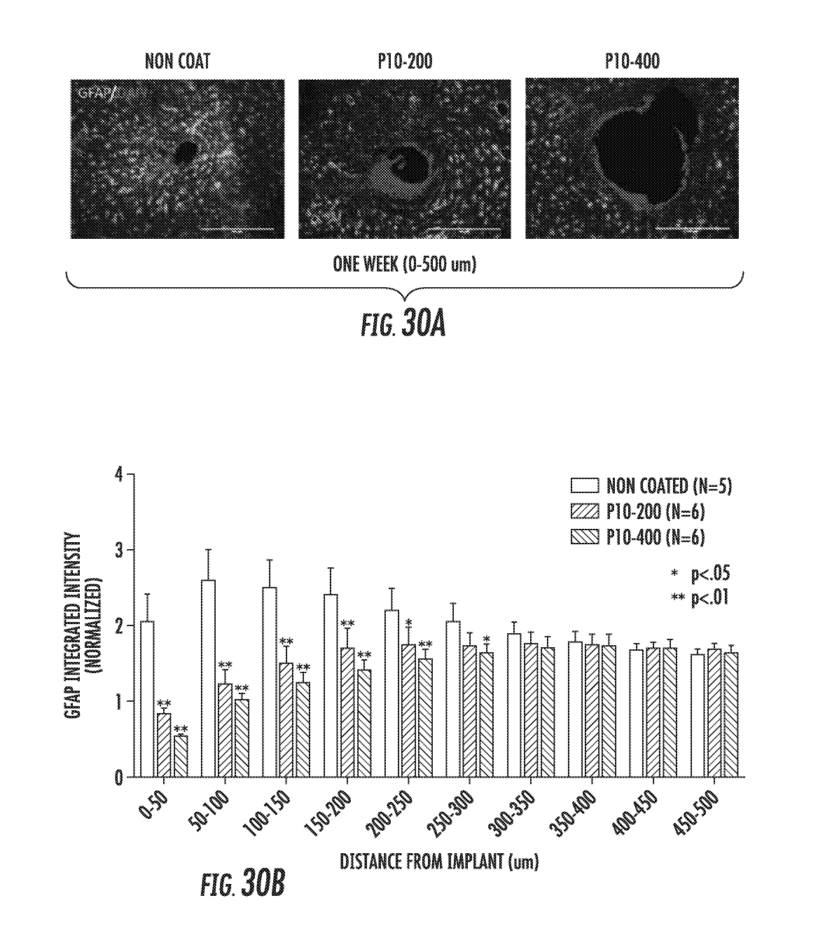

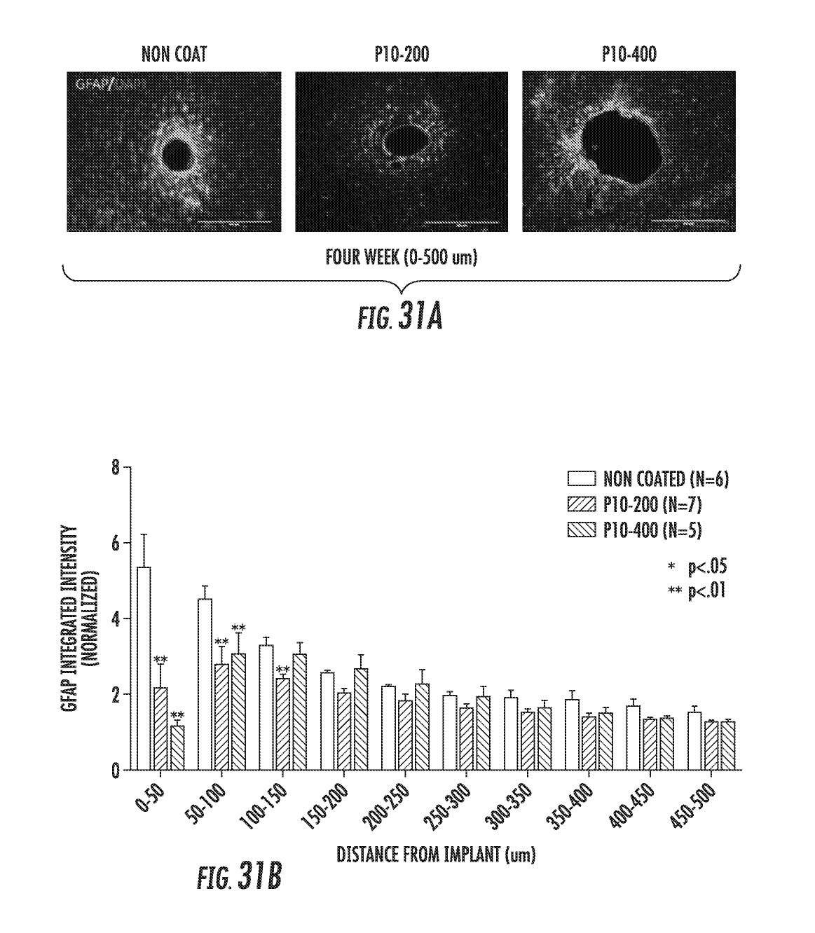

Coated and uncoated devices were implanted in the rodent brain in order to assess the ability to reduce scar formation in vivo. Borosilicate glass neural implant (150 μm in diameter) was coated with a PEG-DA hydrogel of two thicknesses (200 μm and 400 μm total thickness after hydrogel swollen). At 1 week and 4 weeks post implantation, the rodents were sacrificed and brains were sectioned for histology. Tissue sections were stained for the astrocytic glial scar marker GFAP. FIGS. 30A and 31A show representative fluorescent images for the three experimental groups. FIGS. 30B and 31B show quantification of the GFAP expression as a function of distance from the implant at 1 and 4 weeks post implantation, respectively. There was a statistically significant reduction in the GFAP response for both hydrogel coated samples compared to controls.

Although specific embodiments of the disclosure have been described, numerous other modifications and alternative embodiments are within the scope of the disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments.

We claim:

1. A medical device for use in brain tissue, comprising:
a neural implant configured for insertion into brain tissue; and
a coating disposed on a surface of the neural implant,
wherein the coating is configured to exhibit in vivo (i) an elastic modulus that substantially matches the elastic modulus brain tissue and (ii) a thickness to accommodate micromotions of the brain tissue relative to the neural implant.

2. The device of claim 1, wherein the coating has an elastic modulus of about 5 kPa to about 300 kPa.

3. The device of claim 1, wherein the thickness of the coating in vivo is from about 15 microns to about 500 microns.

4. The device of claim 1, wherein the coating comprises a hydrogel.

5. The device of claim 1, wherein the coating is covalently bound to the surface of the neural implant.

6. The device of claim 1, wherein the coating is dry prior to insertion of the neural implant into the brain tissue and rehydrates and swells following insertion of the neural implant into the brain tissue.

7. The device of claim 1, wherein the coating comprises a PEG hydrogel.

8. The device of claim 7, wherein the swollen coating has an elastic modulus from 5 kPa to 300 kPa and a thickness from about 30 microns to about 500 microns.

9. The device of claim 1, wherein the neural implant is configured to provide chemical stimulation, electrical stimulation, sensing of neural activity, or a combination thereof.

10. A medical device comprising:
a neural implant configured for insertion into brain tissue; and
a coating disposed on a surface of the neural implant,
wherein the coating has one or more mechanical properties that substantially match one or more mechanical properties of brain tissue, such that, when the neural implant is implanted into the brain, the coating is effective to reduce scar formation about the neural implant, the scar formation being due at least in part to movement of the brain tissue relative the neural implant and the reduction in scar formation being relative to the scar formation that would be formed on a neural implant without the coating.

11. A method to reduce scar formation about a neural implant due to brain tissue and neural implant movement, the method comprising:
coating a surface of the neural implant with a coating material that comprises one or more mechanical properties that substantially match one or more mechanical properties of the brain tissue, before positioning the neural implant in the brain tissue.

12. The method of claim 11, wherein the coating material is also dried before positioning the neural implant in the brain tissue.

13. The method of claim 11, further comprising covalently bonding the coating material to a surface of the neural implant.

14. The method of claim 11, wherein coating material comprises a polymer and the step of coating further comprises crosslinking the polymer to achieve a selected elastic modulus of the coating material.

15. The method of claim 11, wherein the coating is applied to the surface of the neural implant by spray coating, cast molding, dip coating, or a combination thereof.

16. The method of claim 11, wherein the coating material comprises a hydrogel and, after positioning the neural implant in the brain tissue, has an elastic modulus of about 5 kPa to about 300 kPa and a thickness from about 15 microns to about 500 microns.

17. The method of claim 16, wherein the coating material has a thickness from about 100 microns to about 500 microns.

18. A system for delivering chemical and/or electrical stimulation across one or more neural circuits and/or to sense and record neural activity, the system comprising:
an elongated, rigid neural implant insertable into the brain tissue; and
a hydrogel coating disposed on a surface of the neural implant,
wherein the hydrogel coating which is configured to exhibit in vivo (i) an elastic modulus that substantially matches the elastic modulus brain tissue and (ii) a thickness to accommodate micromotions of the brain tissue relative to the neural implant.

19. The system of claim 18, wherein the hydrogel coating comprises an elastic modulus of about 5 kPa to about 300 kPa.

20. The system of claim 18, wherein the hydrogel coating has a thickness of from about 15 micron to about 500 microns.

21. The system of claim 18, wherein the surface of the neural implant is cylindrical and the hydrogel coating covers the full circumference of the cylindrical surface about substantially the full length of the neural implant insertable into the brain tissue.
